(19) United States
(12) Patent Application Publication
(10) Pub. No.: US 2009/0062517 A2
(43) Pub. Date: Mar. 5, 2009

(54) Dissymmetrical Diazo Compounds Containing Having at Least One 2-imidazolium Unit and a Cationic or Non-cationic Linker, Compositions Comprising Them, Method of Coloring, and Device

(75) Inventors: Herve DAVID, la Varenne Saint Hilaire (FR); Andrew GREAVES, Montevrain (FR); Nicolas DAUBRESSE, la Celles St Cloud (FR)

Correspondence Address: FINNEGAN, HENDERSON, FARABOW, GARRETT & DUNNER;LLP, 901 NEW YORK AVENUE, NW, WASHINGTON, DC, 20001-4413, UNITED STATES, (202) 408-4000

(73) Assignee: L'Oreal S.A., Paris, FR (FR)

(21) Appl. No.: 11/300,284

(22) Filed: Dec. 15, 2005

Related U.S. Application Data
(60) Provisional application No. 60/646,975, filed on Jan. 27, 2005.

(30) Foreign Application Priority Data
Dec. 15, 2004 (FR) 0453000

(51) Int. Cl. C07D 403/02

(52) U.S. Cl. 534/574

(57) ABSTRACT

The present disclosure relates to dissymmetrical cationic diazo compounds having at least one 2-imidazolium unit and a cationic or non-cationic linker. The disclosure further relates to dyeing compositions comprising such compounds as a direct dye in a medium appropriate for the dyeing of keratin fibers, and also to a method of coloring keratin fibers that employs this composition, and to a device having a plurality of compartments.

This application claims benefit of U.S. Provisional Application No. 60/646,975, filed Jan. 27, 2005, the contents of which are incorporated herein by reference. This application also claims benefit of priority under 35 U.S.C. § 119 to French Patent Application No. 04 53000, filed Dec. 15, 2004, the contents of which are also incorporated herein by reference.

The present disclosure relates to dissymmetrical cationic diazo compounds having at least one 2-imidazolium unit and a cationic or non-cationic linker, to dyeing compositions comprising such compounds as a direct dye in a medium appropriate for the dyeing of keratin fibers, to a method of coloring keratin fibers that employs this composition, and to a device having a plurality of compartments.

It is known practice to dye keratin fibers, including human keratin fibers such as the hair, with dyeing compositions containing direct dyes. These compounds are colored, and coloring, molecules having an affinity for the fibers. It is known practice, for example, to use direct dyes of nitrobenzene type, anthraquinone dyes, nitropyridines and dyes of azo, xanthene, acridine, azine or triarylmethane type.

Commonly these dyes are applied to the fibers, optionally in the presence of an oxidizing agent if a simultaneous fiber lightening effect is desired. When the leave-in time has elapsed, the fibers are rinsed, optionally washed, and dried.

The colorations which result from the use of direct dyes are temporary or semi-permanent colorations, because the nature of the interactions which bind the direct dyes to the keratin fiber, and their desorption from the surface and/or the core of the fiber, are responsible for their relatively low tinctorial strength and relatively poor wash resistance or perspiration resistance.

It is known from European Patent Application No. EP 1377263 to employ particular direct cationic diazo dyes containing two cationic heterocyclic groups. These compounds, although representing an advance in the art, give dyeing results which nevertheless remain capable of improvement.

For the purposes of the present disclosure, and in the absence of any indication otherwise:
An alkyl(ene) radical or the alkyl(ene) moiety of a radical is linear or branched.
An alkyl(ene) radical or the alkyl(ene) moiety of a radical is said to be substituted when it comprises at least one substituent selected from the following groups:
   hydroxyl;
   C₁-C₄ alkoxy, C₂-C₄ (poly)hydroxyalkoxy;
   amino, amino substituted by one or two identical or different C₁-C₄ alkyl groups which optionally carry at least one hydroxyl or C₁-C₂ alkoxy group, it being possible for said alkyl radicals to form, with the nitrogen atom to which they are attached, a heterocycle containing 5 or 7 ring members which is saturated or unsaturated, is optionally aromatic, is optionally substituted and optionally contains at least one other heteroatom chosen from nitrogen or another heteroatom;
   an alkylcarbonylamino radical (R′CO—NR—) in which the radical R is chosen from a hydrogen atom and a C₁-C₄ alkyl radical;
   an alkylsuiphonyl radical (R—SO₂—) in which the radical R is a C₁-C₄ alkyl radical;
   an alkylsulphinyl radical (R—SO—) in which the radical R is a C₁-C₄ alkyl radical; and
   an alkylcarbonyl radical (R—CO—) in which the radical R is a C₁-C₄ alkyl radical.
An aromatic or non-aromatic, saturated or unsaturated (hetero)cyclic radical, or the aromatic or non-aromatic, saturated or unsaturated (hetero)cyclic moiety of a radical, is said to be substituted when it comprises at least one substituent, for example carried by a carbon atom, chosen from:
   an optionally substituted C₁-C₁₆ alkyl radical, such as, for example, a C₁-C₈ alkyl radical;
   a halogen atom such as, for example, chlorine, fluorine or bromine;
   a hydroxyl group;
   a C₁-C₄ alkoxy radical; a C₂-C₄ (poly)hydroxyalkoxy radical;
   an amino radical;
   an amino radical substituted by one or two identical or different C₁-C₄ alkyl radicals which optionally carry at least one hydroxyl or amino or C₁-C₄ (mono- or di-)alkylamino or C₁-C₂ alkoxy group, it being possible for the two alkyl radicals, with the nitrogen atom to which they are attached, to form a heterocycle containing 1 to 3 heteroatoms, such as, for example, 1 or 2 heteroatoms, chosen from N, O and S, such as, for example, N, the heterocycle containing 5 to 7 ring members, being saturated or unsaturated and aromatic or non-aromatic, and optionally being substituted;
   an alkylcarbonylamino radical (R′CO—NR—) in which the radical R is chosen from a hydrogen atom and a C₁-C₄ alkyl radical and the radical R′ is a C₁-C₂ alkyl radical;
   an aminocarbonyl radical ((R)₂N—CO—) in which the radicals R, which are identical or different, are chosen from a hydrogen atom and a C₁-C₄ alkyl radical;
   an alkylsulphonylamino or aryisulphonylamino radical (R′SO₂—NR—) in which the radical R is chosen from a hydrogen atom and a C₁-C₄ alkyl radical and the radical R′ is chosen from a C₁-C₄ alkyl radical and a phenyl radical; and
   an aminosulphonyl radical ((R)₂N—SO₂—) in which the radicals R, which are identical or different, are chosen from a hydrogen atom and a C₁-C₄ alkyl radical.

The compounds according to the present disclosure are termed “dissymmetrical” when there is no plane of symmetry perpendicular to the linker L. In other words, the two formula members on either side of the linker L are different. More specifically, they are different when their substituents differ in terms of their identities and/or their positions in the molecule.

Where the different groups forming part of the structure of the compounds according to the present disclosure are substituted, the skilled person will select them such that the dissymmetry of the molecule is respected. One aspect of the present disclosure relates to direct dyes which do not exhibit one or more of the drawbacks of existing direct dyes.

The present disclosure accordingly provides dissymmetrical cationic diazo compounds represented by formulae (I), (II), (III) and (IV) below, their resonance forms, and their acid addition salts and/or their solvates, wherein in the compounds of formulae (I), (II), (III) and (IV), the formula members attached to each side of the linker L are either compositionally different or positionally different such that the compounds as a whole are dissymmetrical: 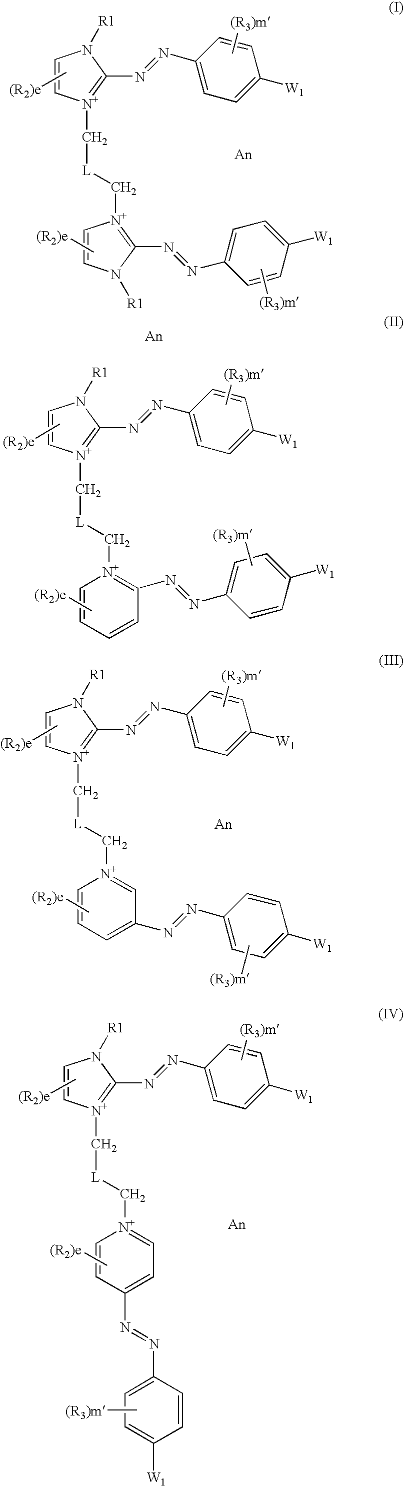
in which formulae:
the radicals R₁, which are identical or different, independently of one another may be chosen from:
optionally substituted C₁-C₄ alkyl radicals;
optionally substituted phenyl radicals; and
optionally substituted benzyl radicals;
   the radicals R₂, which are identical or different, independently of one another may be chosen from:
optionally substituted C₁-C₁₆ alkyl radicals optionally interrupted by at least one heteroatom and/or by at least one group containing at least one heteroatom, chosen from, for example, oxygen, nitrogen, sulphur, —CO—, —SO₂— or combinations thereof, said alkyl radical being further optionally substituted by at least one group chosen from thio (—SH), C₁-C₄ thioalkyl; C₁-C₄ alkylsulphinyl and C₁-C₄ alkylsulphonyl groups;
hydroxyl groups,
C₁-C₄ alkoxy groups,
C₂-C₄ (poly)hydroxyalkoxy groups;
alkoxycarbonyl groups (RO—CO—) wherein R is a C₁-C₄ alkyl radical;
alkylcarbonyloxy radicals (RCO—O—) wherein R is a C₁-C₄ radical;
alkylcarbonyl radicals (R—CO—) wherein R is a C₁-C₄ alkyl radical;
amino groups;
amino groups substituted by one or two identical or different C₁-C₄ alkyl radicals optionally carrying at least one hydroxyl group, it being possible for the two alkyl radicals optionally to form, with the nitrogen atom to which they are attached, a heterocycle containing 1 to 3 heteroatoms, such as, for example, 1 to 2 heteroatoms, chosen from N, O and S, such as, for example, N, and containing 5 to 7 ring members, wherein said heterocycle is saturated or unsaturated, aromatic or non-aromatic and is optionally substituted;
alkylcarbonylamino groups (RCO—NR′—) wherein the radical R is a C₁-C₄ alkyl radical and the radical R′ is chosen from hydrogen and a C₁-C₄ alkyl radical;
aminocarbonyl groups ((R)₂N—CO—) wherein the radicals R independently of one another are chosen from a hydrogen atom and a C₁-C₄ alkyl radical;
ureido groups (N(R)₂—CO—NR′—) wherein the radicals R and R′ independently of one another are chosen from a hydrogen atom and a C₁-C₄ alkyl radical;
aminosulphonyl groups ((R)₂N—SO₂—) wherein the radicals R independently of one another are chosen from a hydrogen atom and a C₁-C₄ alkyl radical;
alkylsulphonylamino groups (RSO₂—NR′—) wherein R is a C₁-C₄ alkyl radical and R′ is chosen from a hydrogen atom and a C₁-C₄ alkyl radical;
optionally substituted aryl radicals;
optionally substituted (C₁-C₄ alkyl)aryl radicals;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms, such as, for example, chlorine or fluorine;
thio groups (HS—);
alkylthio groups (RS—) wherein the radical R is an optionally substituted C₁-C₄ alkyl radical;
when e is 2, for each of the formulae (I), (II), (III), (IV), two radicals R₂ may optionally form, with the carbon atoms to which they are attached, a secondary ring, aromatic or non-aromatic, containing 5 or 6 ring members, such as, for example, 6 members, which is optionally substituted by one or more identical or different groups chosen from hydroxyl, C₁-C₄ alkyl, C₁-C₄ alkoxy, C₂-C₄ (poly)hydroxyalkoxy, amino, and amino substituted by one or two identical or different C₁-C₄ alkyl radicals which optionally carry at least one hydroxyl group;
   e is an integer ranging from 0 to 4; when e is less than 4, the unsubstituted carbon atom(s) of the heterocycle of the formulae (I), (II), (III) and (IV) carry a hydrogen atom, the radicals R₃, from the formulae (I), (II), (III) and (IV), which are identical or different, independently of one another may be chosen from:
optionally substituted C₁-C₁₆ alkyl radicals optionally interrupted by at least one heteroatom or by at least one group containing at least one heteroatom chosen from, for example, oxygen, nitrogen, sulphur, —CO—, —SO₂— or combinations thereof;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group; it being possible for the two alkyl radicals optionally to form, with the nitrogen atom to which they are attached, a heterocycle containing 1 to 3 heteroatoms, such as, for example, 1 to 2 heteroatoms, chosen from N, O and S, such as, for example, N, and containing 5 to 7 ring members, which is saturated or unsaturated, aromatic or non-aromatic and is optionally substituted;

alkylcarbonylamino groups (RCO—NR′—) wherein the radical R is a C₁-C₄ alkyl radical and the radical R′ is chosen from a hydrogen atom and a C₁-C₄ alkyl radical;
aminocarbonyl groups ((R)₂N—CO—) wherein the radicals R independently of one another are chosen from a hydrogen atom and a C₁-C₄ alkyl radical;
ureido groups (N(R)₂—CO—NR′—) wherein the radicals R and R′ independently of one another are chosen from a hydrogen atom and a C₁-C₄ alkyl radical;
aminosulphonyl groups ((R)₂N—SO₂—) wherein the radicals R independently of one another are chosen from a hydrogen atom and a C₁-C₄ alkyl radical;
alkylsulphonylamino groups (RSO₂—NR′—) wherein the radicals R and R′ independently of one another are chosen from a hydrogen atom and a C₁-C₄ alkyl radical;
thio groups (HS—);
alkylthio groups (RS—) wherein the radical R is a C₁-C₄ alkyl radical;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms, such as, for example, chlorine or fluorine;
when m′ is greater than or equal to 2, two adjacent radicals R₃ may form, with the carbon atoms to which they are attached, a secondary ring, aromatic or non-aromatic containing 6 ring members, which is optionally substituted by one or more identical or different groups chosen from the following groups: hydroxyl, C₁-C₄ alkyl, C₁-C₄ alkoxy, C₂-C₄ (poly)hydroxyalkoxy, amino, and amino substituted by one or two identical or different C₁-C₄ alkyl radicals which optionally carry at least one hydroxyl group,
   m′ is an integer ranging from 0 to 4; when m′ is less than 4, then the unsubstituted carbon atom(s) of the aromatic ring of the formulae (I), (II), (III) and (IV) carry a hydrogen atom;
   The W₁ groups of the formulae (I), (II), (III) and (IV), which are identical or different, independently of one another, may be chosen from:
hydrogen atoms;
halogen atoms chosen from bromine, chlorine and fluorine, such as, for example, chlorine and fluorine;
an —NR₅R₆, OR₇, —NR₄-Ph-NR₅R₆, —NR₄-Ph-OR₇, —O-Ph-OR₇ or —O-Ph-NR₅R₆ group, wherein:
   R₄ and R₇, which are identical or different, may be chosen from hydrogen atoms, optionally substituted C₁-C₂₀ alkyl radicals, such as, for example, an optionally substituted C₁-C₁₆ alkyl radical, optionally substituted C₁-C₃ aralkyl radicals and optionally substituted phenyl radicals;
   R₅ and R₆, which are identical or different, may be chosen from hydrogen atoms, optionally substituted C₁-C₂₀ alkyl radicals, such as, for example, an optionally substituted C₁-C₁₆ alkyl radical, optionally substituted phenyl radicals, optionally substituted C₁-C₃ aralkyl radicals and alkylcarbonyl radicals (R—CO—) wherein R is a C₁-C₄ alkyl radical;
   R₅ and R₆ may optionally form, with the nitrogen atom to which they are attached, a heterocycle containing 1 to 3 heteroatoms, such as, for example, 1 or 2 heteroatoms, chosen from N, O and S, such as, for example, N, and containing 5 to 7 ring members, which is saturated or unsaturated, aromatic or non-aromatic and is optionally substituted;
   R₅ and R₆ may optionally form, with the carbon atom of the aromatic ring adjacent to that to which —NR₅R₆ is attached, a 5- or 6-membered saturated heterocycle; and
   Ph is an optionally substituted phenyl radical;
      L is a cationic or non-cationic linker;
      the electroneutrality of the compounds of formula (I), (II), (III) and (IV) being ensured by one or more identical or different, cosmetically acceptable anions An.

The present disclosure also relates to dyeing compositions comprising such compounds of structure (I), (II), (III) and (IV), or their addition salts with an acid, as direct dyes in a medium appropriate for the dyeing of keratin fibers.

A further aspect of the present disclosure relates to a method of coloring keratin fibers which comprises contacting a composition according to the present disclosure with said fibers, which are dry or wet, for a time sufficient to give a desired effect, i.e., desired color.

The present disclosure also relates to a device having a plurality of compartments, i.e., a multi-compartment device, and containing in a first compartment the composition according to the present disclosure and in a second compartment an oxidizing composition.

It has been found that the compounds of formula (I) as defined above may exhibit effective resistance to external agents such as, for example, shampoos, and may do so even when the keratin fiber is sensitized. Furthermore, these compounds may exhibit improved dyeing properties, such as the chromaticity, the coloring power, and/or a low selectivity, which is to say that the compounds of the present disclosure may allow colorations to be obtained which are more uniform between the end and the root of the hair.

Other characteristics and advantages of the present disclosure, however, will appear more clearly from reading the description and the examples presented hereinbelow.

In the text below, and in the absence of any indication otherwise, the end-points delimiting a range of values are included in that range.

As indicated above, the present disclosure relates to compounds corresponding to the formulae (I), (II), (III) and (IV), their salts and/or solvates.

In one embodiment, the compounds of formulae (I), (II), (III) and (IV) according to the present disclosure are such that the identical or different radicals R₁ independently of one another may be chosen from:
C₁-C₄ alkyl radicals which are optionally substituted by at least one radical chosen from hydroxyl, C₁-C₂ alkoxy, amino and C₁-C₂ (di)alkylamino radicals;
optionally substituted benzyl radicals.

According to one embodiment, the identical or different groups R₁ may be chosen from methyl, ethyl, 2-hydroxyethyl, 2-methoxyethyl and benzyl radicals.

The compounds of formula (I), (II), (III) and (IV) according to the present disclosure may be such that the radicals R₂, which are identical or different, independently of one another may be chosen from:
halogen atoms chosen from chlorine and fluorine;
C₁-C₄ alkyl radicals optionally substituted by one or more identical or different radicals chosen from hydroxyl, C₁-C₂ alkoxy, C₂-C₄ (poly)hydroxyalkoxy, amino, C₁-C₂ (di)alkylamino, thio (—SH), C₁-C₄ alkylsulphinyl, C₁-C₄ alkylsulphonyl and C₁-C₄ thioalkyl radicals;
phenyl radicals optionally substituted by at least one identical or different radicals chosen from hydroxyl, C₁-C₂ alkoxy, C₂-C₄ (poly)hydroxyalkoxy, amino and C₁-C₂ (di)alkylamino radicals and a halogen atom such as, for example, chlorine or fluorine;
C₁-C₄ alkoxy radicals;
C₁-C₄ alkylsulphonylamino radicals;
C₂-C₄ (poly)hydroxyalkoxy radicals; amino radicals;
C₁-C₂ (di)alkylamino radicals; C₂-C₄ (poly)hydroxyalkylamino radicals;
alkylsulphonylamino radicals (RSO₂N—) wherein the radical R is C₁-C₄ alkyl radical;
aminosulphonyl radicals ((R)₂NSO₂—) wherein the radicals R independently of one another are chosen from a hydrogen atom and a C₁-C₄ alkyl radical;
alkylthio radicals (RS—) wherein the radical R is a C₁-C₄ alkyl radical;
alkylsulphinyl radicals (RSO—) wherein the radical R is a C₁-C₄ alkyl radical;
alkylsulphonyl radicals (R—SO₂—) wherein the radical R is a C₁-C₄ alkyl radical;
alkylcarbonylamino radicals (RCONR′—) wherein the radical R is chosen from a hydrogen atom and a C₁-C₄ alkyl radical and the radical R′ is chosen from a hydrogen atom and a C₁-C₄ alkyl radical.

According to one embodiment, the identical or different radicals R₂ from formulae (I), (II), (III) and (IV) independently of one another may be chosen from methyl, ethyl, 2-hydroxyethyl, 2-methoxyethyl, methylsulphonyl (CH₃SO₂—), methyl-carbonylamino (CH₃CONH—), hydroxyl, amino, methylamino, dimethylamino, 2-hydroxyethylamino, methoxy, ethoxy and phenyl radicals.

According to a further embodiment, the two radicals R₂ from formulae (I), (II), (III) and (IV) may optionally form, with the carbon atoms to which they are attached, a secondary, 6-membered aromatic ring optionally substituted by at least one identical or different group chosen from hydroxyl, C₁-C₄ alkyl, C₁-C₄ alkoxy, amino, and amino substituted by one or two identical or different C₁-C₄ alkyl radicals which optionally carry at least one hydroxyl or methylcarbonylamino group.

In accordance with this further embodiment, the two radicals R₂ may optionally form, with the carbon atoms to which they are attached, a secondary, 6-membered aromatic ring optionally substituted by at least one identical or different group chosen from hydroxyl, methoxy, ethoxy, amino, 2-hydroxyethylamino, dimethylamino and (di)-2-hydroxyethylamino.

According to at least one embodiment, the coefficient e is 0.

According to an embodiment of the present disclosure, the radicals R₃ of the formulae (I), (II), (III) and (IV), which are identical or different, independently of one another may be chosen from:
optionally substituted C₁-C₁₆ alkyl radicals, such as for example, an optionally substituted C₁-C₈ alkyl radical;
halogen atoms such as, for example, chlorine or fluorine;
hydroxyl groups;
C₁-C₂ alkoxy radicals; C₂-C₄ (poly)hydroxyalkoxy radicals;
amino radicals;
amino radicals substituted by one or two identical or different C₁-C₄ alkyl radicals which optionally carry one or more identical or different groups chosen from hydroxyl and C₁-C₄ alkoxy, it being possible for the two alkyl radicals to form, with the nitrogen to which they are attached, a heterocycle containing 1 to 3 heteroatoms, such as, for example, 1 or 2 heteroatoms, chosen from N, O and S, such as, for example, N, the heterocycle containing 5 to 7 ring members, being saturated or unsaturated, aromatic or non-aromatic, and being optionally substituted;
alkylcarbonylamino radicals (RCO—NR′—) wherein the radical R is a C₁-C₄ alkyl radical and the radical R′ is chosen from a hydrogen and a C₁-C₄ radical;
alkylsulphonylamino radicals (R′SO₂—NR—) wherein the radical R is chosen from a hydrogen atom and a C₁-C₄ alkyl radical and the radical R′ is a C₁-C₄ alkyl radical;
aminosulphonyl radicals ((R)₂N—SO₂—) wherein the radicals R, which are identical or different, are chosen from a hydrogen atom and a C₁-C₄ alkyl radical;
alkylthio radicals (RS—) wherein the radical R is a C₁-C₄ alkyl radical; and
alkylsulphonyl radicals (R—SO₂—) wherein the radical R is a C₁-C₄ alkyl radical.

In at least one embodiment the radicals R₃, which are identical or different, independently of one another, may be chosen from:
C₁-C₄ alkyl radicals optionally substituted by at least one identical or different radicals chosen from hydroxyl radicals, C₁-C₂ alkylcarbonylamino radicals, amino radicals substituted by two identical or different C₁-C₂ alkyl radicals which optionally carry one or more identical or different groups chosen from hydroxyl or C₁-C₂ alkoxy, it being possible for these two alkyl radicals optionally to form, with the nitrogen atom to which they are attached, a 5- or 6-membered heterocycle which is saturated or unsaturated and is optionally aromatic, chosen from, for example, pyrrolidine, piperazine, homopiperazine, pyrrole, imidazole and pyrazole;
C₂-C₄ hydroxyalkoxy radicals; halogen atoms chosen from chlorine and fluorine;
amino radicals;
amino radicals substituted by one or two identical or different C₁-C₂ alkyl radicals which optionally carry at least one hydroxyl group;
methylcarbonylamino radicals;
methylsulphonylamino radicals; hydroxyl radicals;
C₁-C₂ alkoxy radicals;
methylsulphonyl radicals.

According to this embodiment, the radicals R₃, which are identical or different, independently of one another, may be chosen from, for example:
methyl, ethyl, propyl, 2-hydroxyethyl, methoxy, ethoxy, 2-hydroxyethyloxy, 3-hydroxypropyloxy and 2-methoxyethyl radicals;
methylsulphonylamino radicals; amino, methylamino, dimethylamino and 2-hydroxyethylamino radicals;
methylcarbonylamino radicals;
hydroxyl radicals;
chlorine atoms; and
methylsulphonyl radicals.

According to a further embodiment, when the coefficient m′ is greater than or equal to 2, two adjacent radicals R₃ may form, with the carbon atoms to which they are attached, a secondary, 6-membered aromatic ring optionally substituted by one or more identical or different groups chosen from hydroxyl groups, —NR₄—Ph, NR₄-Ph-NR₅R₆ and —NR₄-Ph-OR₇ groups, C₁-C₄ alkyl groups, C₁-C₄ alkoxy groups, C₂-C₄ (poly)hydroxyalkoxy groups, C₁-C₄ alkylcarbonylamino groups, amino groups, and amino groups substituted by one or two identical or different C₁-C₄ alkyl radicals which optionally carry at least one hydroxyl group.

According to this embodiment, two adjacent radicals R₃ may form, with the carbon atoms to which they are attached, a secondary, 6-membered aromatic ring which is optionally substituted by one or more identical or different groups chosen from hydroxyl, methoxy, ethoxy, 2-hydroxyethyloxy, amino, methylcarbonylamino, (di)-2-hydroxyethylamino, —NH-Ph, —NH-Ph-NH₂, —NH-Ph-NHCOCH₃, —NH-Ph-OH and —NH-Ph-OCH₃ groups.

With regard to the radicals R₄ and R₇, these radicals may be chosen from:
hydrogen atoms;
C₁-C₆ alkyl radicals which are optionally substituted by at least one identical or different group chosen from, for example, hydroxyl and C₁-C₂ alkoxy;
aryl and arylalkyl radicals, such as phenyl or benzyl, the aryl moiety being optionally substituted by at least one identical or different group chosen from, for example, a chlorine atom, an amino group, a hydroxyl group, a C₁-C₂ alkoxy group and an amino group which is mono- or disubstituted by two identical or different C₁-C₄ alkyl radicals which optionally carry at least one hydroxyl group.

In accordance with one embodiment of the present disclosure, the radicals R₄ and R₇ may be chosen from:
hydrogen atoms; optionally substituted C₁-C₃ alkyl radicals, such as methyl, ethyl, 2-hydroxyethyl or 2-methoxyethyl;
phenyl radicals which are optionally substituted by at least one identical or different radical chosen from hydroxyl radicals, C₁-C₂ alkoxy radicals, amino radicals, and amino radicals substituted by at least one C₁-C₄ alkyl group which optionally carries at least one hydroxyl group;
   for example, the radicals R₄ and R₇ may be chosen from:
hydrogen atoms;
methyl, ethyl and 2-hydroxyethyl radicals;
phenyl radicals which are optionally substituted by one or more identical or different radicals chosen from hydroxyl, methoxy, amino, (di)methylamino and (di)(2-hydroxyethyl)amino.

With regard to the radicals R₅ and R₆, independently of one another, these identical or different radicals may be chosen from, for example:
hydrogen atoms;
alkylcarbonyl radicals (R—CO—) wherein R is an optionally substituted C₁-C₄ alkyl radical,
C₁-C₆ alkyl radicals which are optionally substituted by at least one identical or different group chosen from hydroxyl, C₁-C₂ alkoxy, amino, C₁-C₄ (di)alkylamino; the alkyl radical may further be substituted by at least one identical or different group chosen from C₁-C₄ alkylsulphonyl, C₁-C₄ alkylsulphinyl and C₁-C₄ alkylcarbonyl,
aryl and arylalkyl radicals, such as phenyl or benzyl, the aryl moiety being optionally substituted by one or more identical or different radicals chosen from, for example, a chlorine atom, an amino group, a hydroxyl group, a C₁-C₄ alkoxy group and an amino group which is mono- or disubstituted by two identical or different C₁-C₄ alkyl radicals which optionally carry at least one hydroxyl group.

In accordance with one embodiment of the invention, the radicals R₅ and R₆, which are identical or different, independently of one another may be chosen from, for example:
hydrogen atoms;
methylcarbonyl, ethylcarbonyl and propylcarbonyl radicals; optionally substituted C₁-C₃ alkyl radicals, such as methyl, ethyl, 2-hydroxyethyl or 2-methoxyethyl;
phenyl radicals which are optionally substituted by at least one identical or different radical chosen from hydroxyl radicals, C₁-C₂ alkoxy radicals, amino radicals, and amino radicals substituted by at least one identical or different C₁-C₄ alkyl group which optionally carries at least one hydroxyl group.

In a further embodiment, the radicals R₅ and R₆, which are identical or different, independently of one another, may be chosen from: hydrogen atoms;
methyl, ethyl and 2-hydroxyethyl radicals;
methylcarbonyl, ethylcarbonyl and propylcarbonyl radicals;
phenyl radicals which are optionally substituted by at least one identical or different radical chosen from hydroxyl, methoxy, amino, (di)methylamino and (di)(2-hydroxyethyl)amino.

In at least embodiment of the present disclosure, the radicals R₅ and R₆ form, together with the nitrogen atom to which each is attached, a heterocycle containing 1 to 3 heteroatoms, such as, for example, 1 or 2 heteroatoms, chosen from N, O and S, such as, for example, N, and containing 5 to 7 ring members, which is saturated or unsaturated, aromatic or non-aromatic, and is optionally substituted.

The heterocycle containing 5 to 7 ring members may be chosen from, for example, the following heterocycles: piperidine, 2-(2-hydroxyethylpiperidine), 4-(aminomethyl)piperidine, 4-(2-hydroxyethyl)piperidine, 4-(dimethylamino)piperidine, piperazine, 1-methyl piperazine, 1-(2-hydroxyethyl)piperazine, 1-(2-aminoethyl)piperazine, 1-hydroxyethylethoxypiperazine, homopiperazine, 1-methyl-1,4-perhydrodiazepine, pyrrole, 1,4-dimethylpyrrole, 1-methyl-4-ethylpyrrole, and 1-methyl-4-propylpyrrole.

In at least one embodiment, the heterocycle containing 5 to 7 ring members may also be chosen from a heterocycle of piperidine, piperazine, homopiperazine, pyrrole, imidazole or pyrazole type which is optionally substituted by one or more identical or different methyl, hydroxyl, amino and/or (di)methylamino radicals

According to another embodiment, the radicals R₅ and R₆ may form, with the carbon atom of the aromatic ring optionally substituted by a hydroxyl and adjacent to that to which —NR₅R₆ is attached, a 5- or 6-membered saturated heterocycle.

For example, the group —NR₅R₆ with the aromatic nucleus optionally substituted by a hydroxyl may correspond to the following compounds: 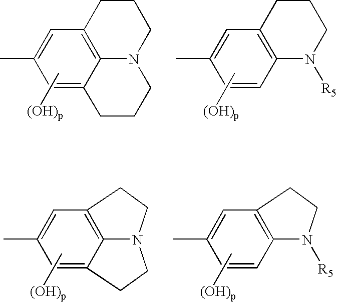
where p=0 or 1.

In a first embodiment, L is a non-cationic linker.

According to this embodiment, non-cationic linker L connecting the two different azo chromophores may be chosen from:
a covalent bond;
optionally substituted C₁-C₄₀ alkyl radicals, such as, for example, an optionally substituted C₁-C₂₀ alkyl radical, optionally interrupted by a saturated or unsaturated aromatic or non-aromatic (hetero)cycle containing 3 to 7 ring members which is optionally substituted and optionally fused, said alkyl radical being optionally interrupted by at least one heteroatom or group containing at least one heteroatom, such as, for example, oxygen, nitrogen, sulphur, —CO—, —SO₂— or combinations thereof, the linker L not containing an azo, nitro, nitroso or peroxo bond;
optionally substituted phenyl radicals.

According to a second embodiment, the linker L is cationic.

According to this embodiment, cationic linker L connecting the two different azo chromophores may be chosen from:
C₂-C₄₀ alkyl radicals which carry at least one cationic charge and are optionally substituted and/or optionally interrupted by at least one saturated or unsaturated, aromatic or non-aromatic, identical or different (hetero)cycle containing 3 to 7 ring members and/or optionally interrupted by at least one heteroatom or group containing at least one heteroatom, or combinations thereof, such as, for example, oxygen, nitrogen, sulphur, a group —CO— or —SO₂— or combinations thereof, the linker L containing no azo, nitro, nitroso or peroxo bond; and with the proviso that the linker L carries at least one cationic charge.

Among non-cationic alkyl-type linkers L that may be used according to the present disclosure, non-limiting mention may be made of methylene, ethylene, linear or branched propylene, linear or branched butylene, linear or branched pentylene, and linear or branched hexylene radicals which are optionally substituted and/or interrupted as indicated above.

These identical or different substituents may be chosen from, for example, hydroxyl, C₁-C₂ alkoxy, C₁-C₂ dialkylamino, (C₁-C₄ alkyl)carbonyl and C₁-C₄ alkyl sulphonyl.

Examples of an aromatic or non-aromatic, saturated or unsaturated cycle or heterocycle interrupting the alkyl radical of the linker L include phenylene, naphthylene, phenanthrylene, triazinyl, pyrimidinyl, pyridinyl, pyridazinyl, quinoxalinyl and cyclohexyl radicals.

Further examples of linkers L include methylene, ethylene, linear or branched propylene, linear or branched butylene, linear or branched pentylene and linear or branched hexylene radicals optionally substituted and/or interrupted as indicated above.

Examples of an aromatic or non-aromatic, saturated or unsaturated cycle or heterocycle interrupting the alkyl radical of the linker L include phenylene or naphthylene, phenanthrylene, triazinyl, pyrimidinyl, pyridinyl, pyridazinyl, quinoxalinyl and cyclohexyl radicals.

Among radicals L that can be used according to an embodiment of the present disclosure, non-limiting mention may be made of the following radicals L:

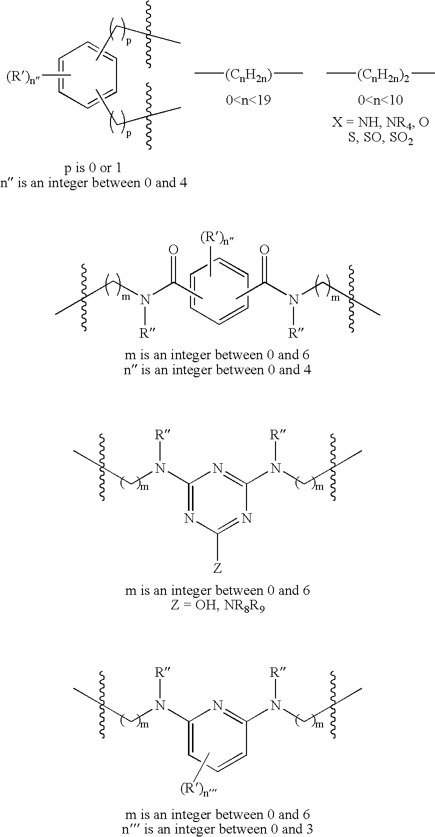

In these formulae:
R′ has the same definition as R₃;
R″ radicals, which are identical, may be chosen from hydrogen and a C₁-C₄ alkyl radical;
R₈ and R₉, independently of one another, may be chosen from a hydrogen atom and a C₁-C₈ alkyl radical which is optionally substituted by one or more identical or different radicals chosen from hydroxyl, C₁-C₂ alkoxy, C₂-C₄ (poly)hydroxyalkoxy, amino, C₁-C₂ (di)alkylamino and optionally substituted aryl.

Examples of radicals L may also include: 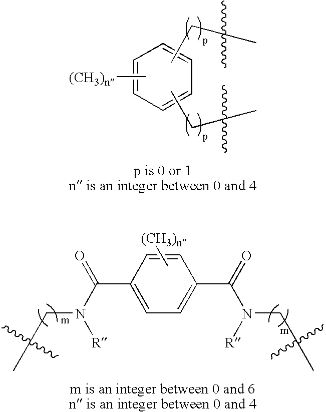
The aromatic ring positions not substituted by a methyl radical carry a hydrogen atom.

Among cationic linker L radicals that my be used according to the present disclosure, non-limiting mention may be made of a C₂-C₂₀ alkyl radical:
1—interrupted by at least one group corresponding to the following forumlae: 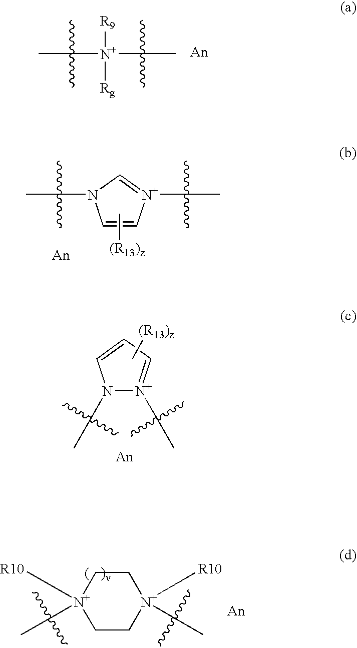
wherein:
R₉ and R₁₀ independently of one another are chosen from C₁-C₈ alkyl radicals; C₁-C₆ monohydroxyalkyl radicals; C₂-C₆ polyhydroxyalkyl radicals; C₁-C₆ alkoxy-C₁-C₆ alkyl radicals; aryl radicals such as phenyl which are optionally substituted; arylalkyl radicals such as benzyl which is optionally substituted; C₁-C₆ aminoalkyl radicals; C₁-C₆ aminoalkyl radicals whose amine is substituted by one or two identical or different C₁-C₄ alkyl radicals; and C₁-C₆ alkylsulphonyl radicals,
two radicals R₉ may together form, with the nitrogen atom to which they are attached, a saturated or unsaturated ring which is optionally substituted and has 5, 6 or 7 ring members,
R₁₃ radicals, which are identical or different, are chosen from halogen atoms chosen from bromine, chlorine and fluorine, C₁-C₆ alkyl radicals, C₁-C₆ monohydroxyalkyl radicals, C₂-C₆ polyhydroxyalkyl radicals, C₁-C₆ alkoxy radicals, C₁-C₄ (di)alkylamino radicals, hydroxycarbonyl radicals, C₁-C₆ alkylcarbonyl radicals, C₁-C₆ thioalkyl radicals, C₁-C₆ alkylthio radicals, C₁-C₆ alkylsulphonyl radicals, optionally substituted benzyl radicals, and phenyl radicals which are optionally substituted by at least one radical chosen from methyl, hydroxyl, amino and methoxy radicals;
An is chosen from an organic anion, an inorganic anion and an anion mixture;
z is an integer ranging from 1 and 3; if z is <3, the unsubstituted carbon atoms carry a hydrogen atom;
v is chosen from an integer 1 and 2, such as, for example, 1;
   2—optionally interrupted by at least one heteroatom or group containing at least one heteroatom, or combinations thereof, such as, for example, oxygen, nitrogen, sulphur, a —CO— group or a —SO₂— group, with the proviso that there is no nitro, nitroso or peroxo bond or group in the linker L; and
   3—optionally substituted by at least one radical chosen from hydroxyl radicals, C₁-C₂ alkoxy radicals, C₂-C₄ (poly)hydroxyalkoxy radicals, amino radicals substituted by at least one linear or branched C₁-C₂ alkyl group which optionally carries at least one hydroxyl group.

According to one embodiment of formulae (a) and (d), R₉ and R₁₀, separately, are chosen from, for example, C₁-C₆ alkyl radicals, C₁-C₄ monohydroxyalkyl radicals, C₂-C₄ polyhydroxyalkyl radicals, C₁-C₆ alkoxy-C₂-C₄ alkyl radicals and C₂-C₆ dimethylaminoalkyl radicals.

In a further embodiment, R₉ and R₁₀ separately are chosen from methyl, ethyl and 2-hydroxyethyl radicals.

According to one embodiment of formulae (b) and (c), R₁₃ is chosen from halogen atoms chosen from chlorine and fluorine, C₁-C₆ alkyl radicals, C₁-C₄ monohydroxyalkyl radicals, C₁-C₄ alkoxy radicals, hydroxycarbonyl radicals, C₁-C₆ alkylthio radicals and amino radicals disubstituted by a C₁-C₄ alkyl radical.

According to at least one embodiment of formulae (b) and (c), R₁₃ is chosen from a chlorine atoms and methyl, ethyl, 2-hydroxyethyl, methoxy, hydroxycarbonyl and dimethylamino radicals.

According to a further embodiment of formulae (b) and (c), z is 0.

In the formulae of the compounds of formulae (I), (II), (III) and (IV), An is chosen from an organic anion, an inorganic anion and an anion mixture allowing the charge or charges on the compounds of formula (I), (II), (III) and (IV) to be balanced, and chosen from, for example, a halide such as chloride, bromide, fluoride or iodide; a hydroxide; a sulphate; a hydrogensulphate; an alkylsulphate for which the linear or branched alkyl moiety is C₁-C₆, such as the methylsulphate or ethylsulphate ion; carbonates and hydrogencarbonates; salts of carboxylic acids, such as formate, acetate, citrate, tartrate and oxalate; alkylsulphonates for which the linear or branched alkyl moiety is C₁-C₆, such as the methylsulphonate ion; arylsulphonates for which the aryl moiety, such as phenyl, is optionally substituted by one or more C₁-C₄ radicals, such as 4-tolylsulphonate, for example; and alkylsulphonyls such as mesylate.

The acid addition salts of the compounds of formulae (I), (II), (III) and (IV) may be, by way of example, the addition salts with an organic or inorganic acid such as hydrochloric acid, hydrobromic acid, sulphuric acid or alkyl or phenyl-sulphonic acids such as p-toluenesulphonic acid or methylsulphonic acid.

The solvates of compounds of formulae (I), (II), (III) and (IV) may be chosen from the hydrates of such compounds and the combination of a compound of formula (I), (II), (III) and (IV) with a linear or branched C₁-C₄ alcohol such as methanol, ethanol, isopropanol or n-propanol.

In accordance with one embodiment of the present disclosure, the compounds correspond to formulae (I′), (I″), (I′″), (II′), (II″), (II′″), (III′), (III″), (III′″), (IV′), (IV″), (IV′″) below, and also to their resonance forms and/or their acid addition salts and/or their solvates: 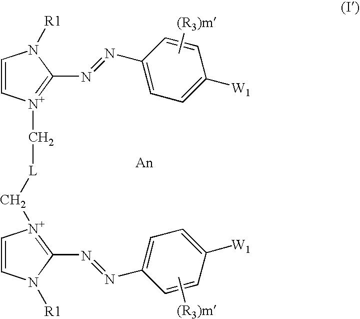
L is a single covalent bond or CnH2n with n being between 1 and 6 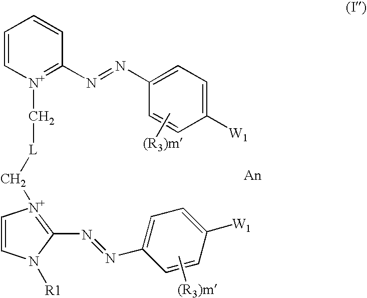
L is a single covalent bond or CnH2n with n being between 1 and 6 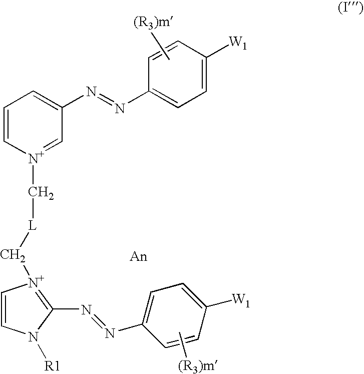
L is a single covalent bond or CnH2n with n being between 1 and 6 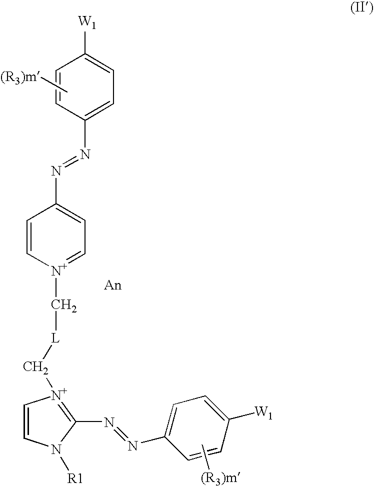
L is a single covalent bond or CnH2n with n being between 1 and 6 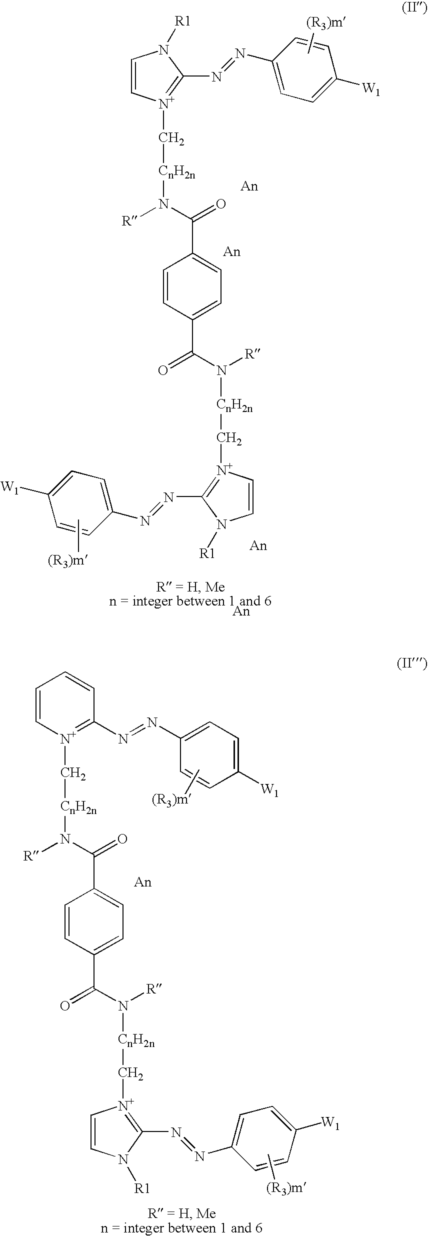 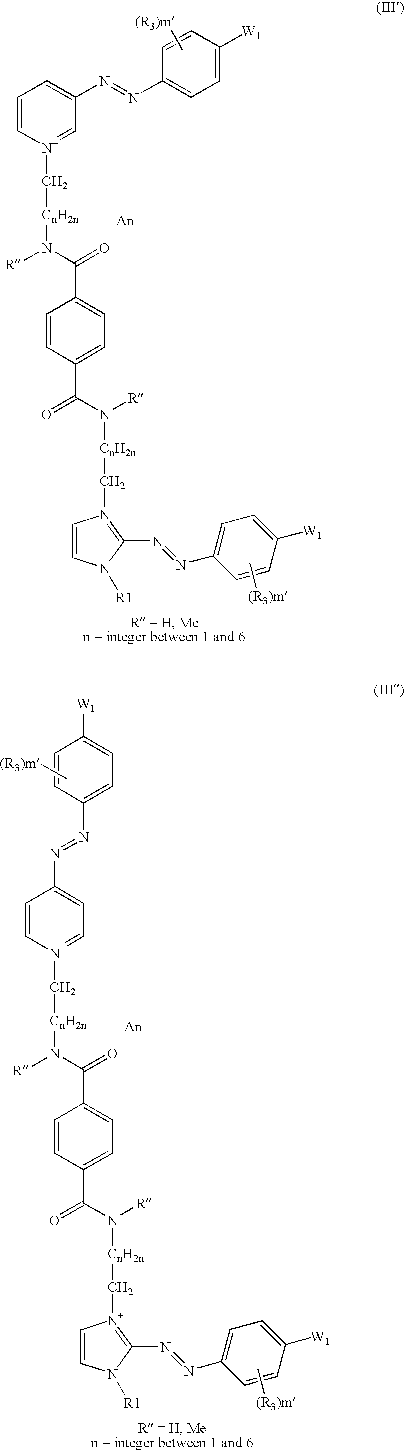
where R₁, R₃ W₁ and m′ are as defined above. 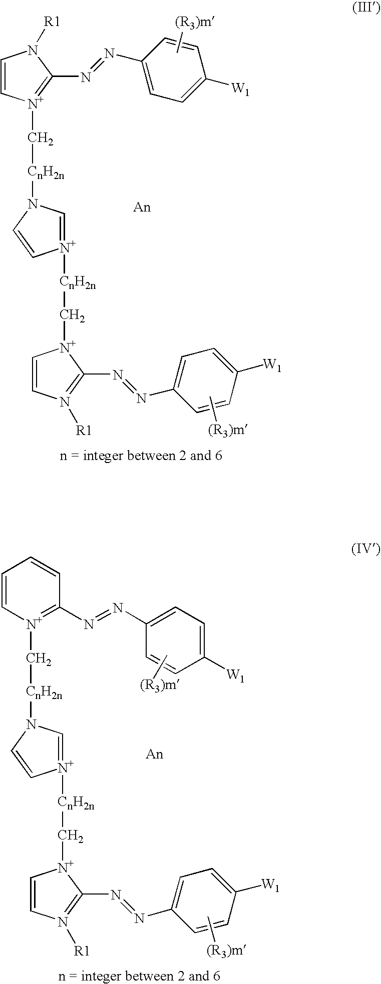 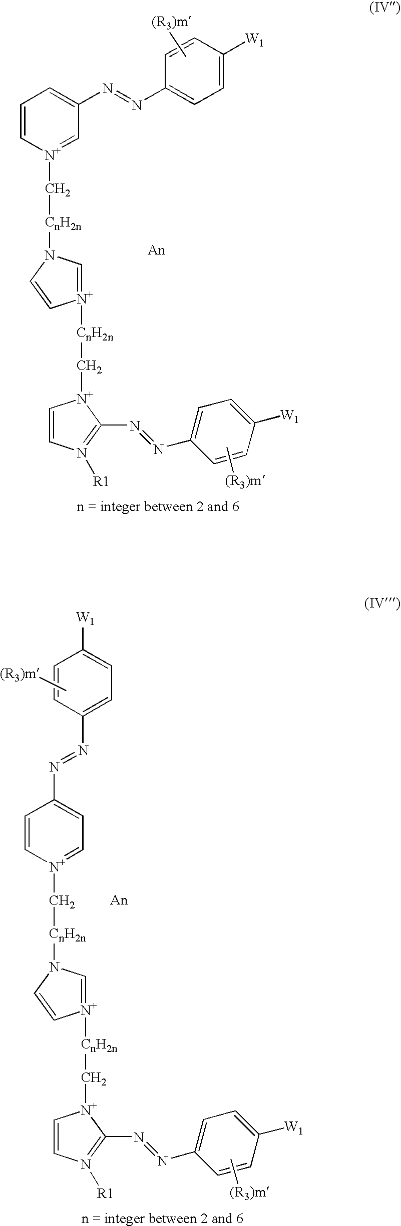
where R₁ R₃, W₁ and m′ are as defined above.

The compounds corresponding to the monoazo species may be obtained from preparation processes described, for example, in the documents U.S. Pat. No. 5,708,151, J. Chem. Res., Synop. (1998), (10), 648-9, U.S. Pat. No. 3,151,106, U.S. Pat. No. 5,852,179, Heterocycles, 1987, 26 (2) 313-7, Synth. Commun. 1999, 29 (13), 2271-6, Tetrahedron, 1983, 39 (7), 1091-1101. As for the diazo compounds, reference may be made to European Patent Application No. EP 1377263 for a synthesis description.

The present disclosure further relates to a dyeing composition comprising at least one compound of formula (I), (II), (III) and/or (IV), or its acid addition salts, as direct dye in a medium appropriate for the dyeing of keratin fibers.

The total concentration of compound(s) of formula (I), (II), (III) and/or (IV) may range from 0.001% to 20% by weight relative to the total weight of the dyeing composition, such as from 0.01% to 10% by weight or from 0.05% to 5% by weight.

The dyeing composition according to the present disclosure may also comprise at least one oxidation base. This oxidation base may be chosen from the oxidation bases conventionally used in oxidation dyeing, such as, for example, para-phenylenediamines, bis(phenyl)alkylenediamines, para-aminophenols, ortho-aminophenois and heterocyclic bases.

Among the para-phenylenediamines that may be used according to at least one embodiment of the present disclosure, non-limiting mention may be made, for example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxy-ethyl)amino-2-methylaniline, 4-N,N-bis(β-hydroxyethyl)amino-2-chloroaniline, 2-p-hydroxyethyl-para-phenylenediamine, 2-fluoro-para-phenylenediamine, 2-isopropyl-para-phenylenediamine, N—(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-hydroxyethyloxy-para-phenylenediamine, 2-β-acetylaminoethyloxy-para-phenylenediamine, N-(β-methoxyethyl)-para-phenylenediamine, 4-amino-phenylpyrrolidine, 2-thienyl-para-phenylenediamine, 2-p-hydroxyethylamino-5-aminotoluene, and the acid addition salts thereof.

Among the para-phenylenediamines mentioned above, para-phenylenediamines chosen from para-phenylenediamine, para-tolylenediamine, 2-isopropyl-para-phenylenediamine, 2-1-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the acid addition salts thereof may be used in at least one embodiment.

Among the bis(phenyl)alkylenediamines that may be used according to the present disclosure, non-limiting mention may be made, for example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-amino-phenyl)tetramethylenediamine, N,N′-bis(β-hydroxyethyl)-N,N′-bis(4-aminophenyl)tetramethylenediamine, N,N′-bis(4-methyl-aminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the acid addition salts thereof.

Among the para-aminophenols that may be used according to the present disclosure, non-limiting mention may be made, for example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the acid addition salts thereof.

Among the ortho-aminophenols that may be used according to the present disclosure, non-limiting mention may be made, for example, of 2-aminophenol, 2-amino-5-methylphenol, 2-amino-6-methylphenol and 5-acetamido-2-aminophenol, and the acid addition salts thereof.

Among the heterocyclic bases that may be used according to the present disclosure, non-limiting mention may be made, for example, of pyridine derivatives, pyrimidine derivatives and pyrazole derivatives.

Among the pyridine derivatives that may be used according to the present disclosure, non-limiting mention may made of the compounds described, for example, in British Patent Nos. GB 1 026 978 and GB 1 153 196, as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the acid addition salts thereof.

Among the pyrimidine derivatives that may be used according to the present disclosure, non-limiting mention may be made of the compounds described, for example, in Patent Nos. DE 2 359 399; JP 88-169 571; JP 05-163124; EP 0 770 375 or International Patent Application No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048 and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]-ethanol, 2-[(7-aminopyrazolo[175-a]pyrimidin-3-yl)(2-hydroxyethyl)amino]ethanol, 5,6-dimethylpyrazolo[1,5-a]pyrimidine-3,7-diamine, 2,6-dimethylpyrazolo-[1,5-a]pyrimidine-3,7-diamine, 2,5,N7,N7-tetramethylpyrazolo[175-a]pyrimidine-3,7-diamine and 3-amino-5-methyl-7-imidazolylpropylaminopyrazolo[1,5-a]pyrimidine, and the acid addition salts thereof and the tautomeric forms thereof, when a tautomeric equilibrium exists.

Among the pyrazole derivatives that may be used according to the present disclosure, non-limiting mention may be made of the compounds described in Patent Nos. DE 3 843 892 and DE 4 133 957 and Patent Application Nos. WO 94/08969, WO 94/08970, FR 2 733 749 A and DE 195 43 988, such as 4,5-diamino-1-methylpyrazole, 4,5-diamino-1-(β-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-methylpyrazole, 4,5-diamino-3-hydroxymethyl-1-isopropylpyrazole, 4,5-diamino-3-methyl-1-isopropylpyrazole, 4-amino-5-(27-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the acid addition salts thereof.

The dyeing composition according to the present disclosure may also contain one or more couplers conventionally used for dyeing keratin fibers. Among these couplers non-limiting mention may be made of meta-phenylenediamines, meta-aminophenols, meta-diphenols, naphthalenic couplers and heterocyclic couplers.

Examples that may also be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene and the acid addition salts thereof.

In the dyeing composition of the present disclosure the oxidation base or bases may be present in a total amount ranging from 0.001% to 10% by weight relative to the total weight of the dyeing composition, such as, for example, from 0.005% to 6% by weight.

The coupler or couplers may be present in a total amount ranging from 0.001% to 10% by weight relative to the total weight of the dyeing composition, such as, for example, from 0.005% to 6% by weight.

The acid addition salts that may be used in the context of the dyeing compositions of the present disclosure for the oxidation bases and couplers may be chosen from those listed in the context of the definition of the compounds of formula (I), (II), (III) and (IV).

The composition according to the present disclosure may optionally comprise at least one additional direct dye other than the compounds of formula (I), (ii), (III) and (IV). This at least one additional direct dye may be chosen from cationic and nonionic species.

Non-limiting examples that may be mentioned include nitrobenzene dyes, azo, azomethine, methine, tetraazapentamethine, anthraquinone, naphthoquinone, benzoquinone, phenothiazine, indigoid, xanthene, phenanthridine and phthalocyanine dyes, dyes derived from triarylmethane, and natural dyes, alone or as mixtures.

The at least one additional direct dye may be chosen from, for example, the following red and orange nitrobenzene dyes:
1-hydroxy-3-nitro-4-N-(γ-hydroxypropyl)aminobenzene;
N-(β-hydroxyethyl)amino-3-nitro-4-aminobenzene;
1-amino-3-methyl-4N-(β-hydroxyethyl)amino-6-nitrobenzene;
1-hydroxy-3-nitro-4-N-(β-hydroxyethyl)aminobenzene;
1,4-diamino-2-nitrobenzene;
1-amino-2-nitro-4-methylaminobenzene;
N-(β-hydroxyethyl)-2-nitro-para-phenylenediamine;
1-amino-2-nitro-4-(β-hydroxyethyl)amino-5-chlorobenzene;
2-nitro-4-aminodiphenylamine;
1-amino-3-nitro-6-hydroxybenzene;
1-(β-aminoethyl)amino-2-nitro-4-(β-hydroxyethyloxy)benzene;
1-(β,γ-dihydroxypropyl)oxy-3-nitro-4-(β-hydroxyethyl)aminobenzene;
1-hydroxy-3-nitro-4-aminobenzene;
1-hydroxy-2-amino-4,6-dinitrobenzene;
1-methoxy-3-nitro-4-(β-hydroxyethyl)aminobenzene;
2-nitro-4′-hydroxydiphenylamine; and
1-amino-2-nitro-4-hydroxy-5-methylbenzene.

The at least one additional direct dye may also be chosen from yellow and green-yellow nitrobenzene direct dyes; non-limiting mention may be made, for example, of the compounds chosen from:
1-β-hydroxyethyloxy-3-methylamino-4-nitrobenzene;
1-methylamino-2-nitro-5-(β,γ-dihydroxypropyl)oxybenzene;
1-(β-hydroxyethyl)amino-2-methoxy-4-nitrobenzene;
1-(β-aminoethyl)amino-2-nitro-5-methoxybenzene;
1,3-di(β-hydroxyethyl)amino-4-nitro-6-chlorobenzene;
1-amino-2-nitro-6-methylbenzene;
1-(β-hydroxyethyl)amino-2-hydroxy-4-nitrobenzene;
N-(β-hydroxyethyl)-2-nitro-4-trifluoromethylaniline;
4-(β-hydroxyethyl)amino-3-nitrobenzenesulphonic acid;
4-ethylamino-3-nitrobenzoic acid;
4-(β-hydroxyethyl)amino-3-nitrochlorobenzene;
4-(β-hydroxyethyl)amino-3-nitromethylbenzene;
4-(β,γ-dihydroxypropyl)amino-3-nitrotrifluoromethylbenzene;
1-(β-ureidoethyl)amino-4-nitrobenzene;
1,3-diamino-4-nitrobenzene;
1-hydroxy-2-amino-5-nitrobenzene;
1-amino-2-[tris(hydroxymethyl)methyl]amino-5-nitrobenzene;
1-(β-hydroxyethyl)amino-2-nitrobenzene; and
4-(β-hydroxyethyl)amino-3-nitrobenzamide.

Mention may also be made of blue and violet nitrobenzene direct dyes; for instance:
1-(β-hydroxyethyl)amino-4-N,N-bis(β-hydroxyethyl)amino-2-nitrobenzene;
1-(β,γ-hydroxypropyl)amino-4-N,N-bis(β-hydroxyethyl)amino-2-nitrobenzene;
1-(β-hydroxyethyl)amino-4-(N-methyl-N-β-hydroxyethyl)amino-2-nitrobenzene;
1-(β-hydroxyethyl)amino-4-(N-ethyl-N-β-hydroxyethyl)amino-2-nitrobenzene;
   1-(β,γ-dihydroxypropyl)amino-4-(N-ethyl-N-β-7-hydroxyethyl)amino-2-nitrobenzene;
   2-nitro-para-phenylenediamines having the following formula: 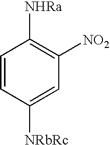
      wherein:
   Rb is chosen from a C₁-C₄ alkyl radical, α-hydroxyethyl, β-hydroxypropyl and γ-hydroxypropyl radical;
   Ra and Rc, which may be identical or different, are chosen from β-hydroxyethyl, γ-hydroxypropyl, y-hydroxypropyl and β,γ-dihydroxypropyl radicals, wherein at least one of the radicals Rb, Rc or Ra is chosen from a γ-hydroxypropyl radical and Rb and Rc are not simultaneously α-hydroxyethyl radical when Rb is a γ-hydroxypropyl radical, such as those described in French Patent No. FR 2 692 572

Among the azo direct dyes that may be used according to the present disclosure, non-limiting mention may be made of the cationic azo dyes described in Patent Application Nos. WO 95/15144, WO 95/01772 and EP 714954, FR 2 822 696, FR 2 825 702, FR 2 825 625, FR 2 822 698, FR 2 822 693, FR 2 822 694, FR 2 829 926, FR 2 807 650, WO 02/078660, WO 02/100834, WO 02/100369 and FR 2 844 269.

Among the azo direct dyes that may be used according to the present disclosure, non-limiting mention may be made of the following dyes:
1,3-dimethyl-2-[[4-(dimethylamino)phenyl]azo]-1H-imidazolium chloride;
1,3-dimethyl-2-[(4-aminophenyl)azo]-1H-imidazolium chloride; and
1-methyl-4-[(methylphenylhydrazono)methyl]pyridinium methylsulphate.

Among the azo direct dyes that may also be mentioned are the following dyes described in the Color Index International 3rd edition:
Disperse Red 17;
Acid Yellow 9;
Acid Black 1;
Basic Red 22;
Basic Red 76;
Basic Yellow 57;
Basic Brown 16;
Acid Yellow 36;
Acid Orange 7;
Acid Red 33;
Acid Red 35;
Basic Brown 17;
Acid Yellow 23;
Acid Orange 24; and
Disperse Black 9.

Non-limiting mention may also be made of 1-(4′-aminodiphenylazo)-2-methyl-4-[bis(β-hydroxyethyl)amino]benzene and 4-hydroxy-3-(2-methoxyphenylazo)-1-naphthalenesulphonic acid.

Among the quinone direct dyes that may be used according to the present disclosure, non-limiting mention may be made of the following dyes:
Disperse Red 15;
Solvent Violet 13;
Acid Violet 43;
Disperse Violet 1;
Disperse Violet 4;
Disperse Blue 1;
Disperse Violet 8;
Disperse Blue 3;
Disperse Red 11;
Acid Blue 62;
Disperse Blue 7;
Basic Blue 22;
Disperse Violet 15;
Basic Blue 99;

and also the following compounds:
1-N-methylmorpholiniumpropylamino-4-hydroxyanthraquinone;
1-aminopropylamino-4-methylaminoanthra-quinone;
1-aminopropylaminoanthraquinone;
5-β-hydroxyethyl-1,4-diaminoanthraquinone;
2-aminoethylaminoanthraquinone; and
1,4-bis(β,γ-dihydroxypropylamino)anthraquinone.

Among the azine dyes that may be used according to the present disclosure, non-limiting mention may be made of the following compounds:
Basic Blue 17; and
Basic Red 2.

Among the triarylmethane dyes that may be used according to the present disclosure non-limiting mention may be made of the following compounds:
Basic Green 1;
Acid Blue 9;
Basic Violet 3;
Basic Violet 14;
Basic Blue 7;
Acid Violet 49;
Basic Blue 26; and
Acid Blue 7.

Among the indoamine dyes that may be used according to the present disclosure, non-limiting mention may be made of the following compounds:
2-β-hydroxyethylamino-5-[bis(β-4′-hydroxyethyl)amino]anilino-1,4-benzoquinone;
2-β-hydroxyethylamino-5-(2′-methoxy-4′-amino)anilino-1,4-benzoquinone;
3-N-(2′-chloro-4′-hydroxy)phenylacetyl-amino-6-methoxy-1,4-benzoquinoneimine;
3-N-(3′-chloro-4′-methylamino)phenylureido-6-methyl-1,4-benzoquinoneimine; and
3-[4′-N-(ethylcarbamyl methyl)amino]phenylureido-6-methyl-1,4-benzoquinoneimine.

Among the dyes of tetraazapentamethine type that may be used according to the present disclosure, non-limiting mention may be made of the following compounds given in the table below, with An being defined as above:

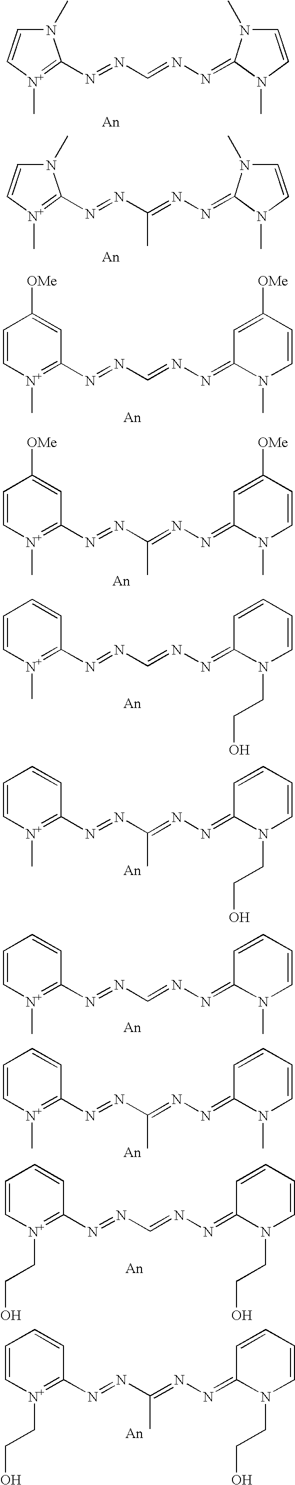

Among the natural direct dyes that may be used according to the present disclosure, non-limiting mention may be made of lawsone, juglone, alizarin, purpurin, carminic acid, kermesic acid, purpurogallin, protocatechaldehyde, indigo, isatin, curcumin, spinulosin and apigenidin. Extracts or decoctions containing these natural dyes may also be used, such as, for example, henna-based poultices and extracts.

When they are present, the amount of additional direct dye in the composition may range from 0.001% to 20% by weight relative to the total weight of the composition, such as from 0.01% to 10% by weight relative to the total weight of the composition.

The medium that is suitable for dyeing, also known as the dye vehicle, generally comprises water or a mixture of water and at least one organic solvent to dissolve the compounds that would not be sufficiently water-soluble.

In one embodiment, the organic solvents may be chosen from, for example, linear or branched saturated monoalcohols or diols containing 2 to 10 carbon atoms, such as ethyl alcohol, isopropyl alcohol, hexylene glycol (2-methyl-2,4-pentanediol), neopentyl glycol and 3-methyl-1,5-pentanediol; aromatic alcohols such as benzyl alcohol and phenylethyl alcohol; glycols or glycol ethers, for instance ethylene glycol monomethyl, monoethyl and monobutyl ether, propylene glycol and its ethers, for instance propylene glycol monomethyl ether, butylene glycol and dipropylene glycol; and also diethylene glycol alkyl ethers, such as the C₁-C₄ ethers, for instance diethylene glycol monoethyl ether or monobutyl ether, alone or as a mixture.

The solvents described above, when they are present, may be present in an amount ranging from 1% to 40% by weight, for example, from 5% to 30% by weight, relative to the total weight of the composition.

The dyeing composition in accordance with the present disclosure may also include various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrants, sequestrants, fragrances, buffers, dispersants, conditioning agents, for instance silicones, which may or may not be volatile or be modified, film-forming agents, ceramides, preservatives and opacifiers.

The adjuvants may be present individually in an amount ranging from 0.01% to 20% by weight relative to the total weight of the composition. The person skilled in the art will of course take care to select this or these optional additional compounds such that the advantageous properties intrinsically associated with the oxidation dyeing composition in accordance with the present disclosure are not, or not substantially, adversely affected by the envisaged addition. The pH of the dyeing composition in accordance with the present disclosure may range from about 3 to 12, such as, for example, from about 5 and 11. The pH may be adjusted to the desired value using acidifying or alkalifying agents usually used in the dyeing of keratin fibers, or alternatively using standard buffer systems.

Among the acidifying agents that may be mentioned, for example, are mineral or organic acids such as hydrochloric acid, orthophosphoric acid, sulphuric acid, carboxylic acids such as acetic acid, tartaric acid, citric acid and lactic acid, and sulphonic acids.

Among the alkalifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds having the following formula: 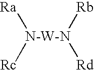
wherein W is a propylene residue optionally substituted by a hydroxyl group or a C₁-C₄ alkyl radical; Ra, Rb, Rc and Rd, which are identical or different, are chosen from hydrogen atoms, C₁-C₄ alkyl radicals and C₁-C₄ hydroxyalkyl radicals. The dyeing composition according to the present disclosure may be in various forms, such as in the form of liquids, creams or gels, or in any other form that is suitable for dyeing keratin fibers, such as human hair.

The composition according to the present disclosure may further comprise at least one oxidizing agent. When the composition comprises at least one oxidizing agent, the composition is referred to as a ready-to-use composition.

For the purposes of the present disclosure, a ready-to-use composition is a composition intended to be applied immediately to the keratin fibers, i.e., it may be stored in unmodified form before use or may result from the extemporaneous mixing of two or more compositions.

The composition may also be obtained by mixing the composition with an oxidizing composition.

The at least one oxidizing agent may be any oxidizing agent conventionally used in the field. Thus it may be chosen from hydrogen peroxide, urea peroxide, alkali metal bromates, persalts such as perborates and persulphates, and also enzymes, among which mention may be made of peroxidases, 2-electron oxidoreductases such as uricases, and 4-electron oxygenases, for instance laccases. In at least one embodiment, hydrogen peroxide may be used as the oxidizing agent. The amount of the at least one oxidizing agent may range from 1% to 40% by weight relative to the total weight of the ready-to-use composition, such as, for example, from 1% to 20% by weight relative to the total weight of the ready-to-use composition.

The oxidizing composition used may be an aqueous composition and may be in the form of a solution or an emulsion.

In at least one embodiment, the composition free of oxidizing agent is mixed with about 0.5 to 10 weight equivalents of the oxidizing composition.

The pH of the ready-to-use composition may range from 4 to 12, such as, for example, from 7 to 11.5.

The pH of the composition may be adjusted using an acidifying or alkalifying agent chosen from those mentioned previously.

The present disclosure further relates to a method of coloring that comprises the application of a dyeing composition according to the present disclosure to wet or dry keratin fibers.

The application to the fibers of the dyeing composition comprising the compound(s) of formula (I), (II), (III) and/or (IV) or the acid addition salts thereof, optionally at least one oxidation base optionally combined with at least one coupler, and optionally at least one additional direct dye, may be performed in the presence of an oxidizing agent.

This oxidizing agent may be added to the composition comprising the compound(s) of formula (I), (II), (III) and/or (IV) and the optional oxidation bases, couplers and/or additional direct dyes, either at the time of use or directly on the keratin fiber.

The oxidizing composition may also include various adjuvants conventionally used in compositions for dyeing the hair and as defined above. The pH of the oxidizing composition containing the oxidizing agent may be such that, after mixing with the dye composition, the pH of the resulting composition applied to the keratin fibers ranges from 4 to 12 approximately, such as, for example, from 7 to 11.5. The pH may be adjusted to the desired value by means of acidifying or alkalifying agents usually used in the dyeing of keratin fibers and as described above.

The composition that is finally applied to the keratin fibers may be in various forms, such as in the form of liquids, creams or gels or in any other form that is suitable for dyeing keratin fibers, such as human hair.

According to one embodiment, the composition according to the present disclosure is free of oxidation base and coupler.

The composition applied may optionally comprise at least one oxidizing agent.

The composition is thus applied to the wet or dry keratin fibers and is then left for a leave-in time that is sufficient to give the desired coloration. Whatever the version adopted (with or without oxidizing agent), the leave-in time may range from a few seconds to one hour, such as, for example, from 3 to 30 minutes.

The temperature at which the composition acts may range from 15 to 220° C., such as, for example, from 15 to 80° C. or from 15 to 40° C. After the leave-in time, the composition may be removed by rinsing with water, optionally followed by washing with a shampoo, and then optionally by drying.

Another aspect of the present disclosure relates to a device having a plurality of compartments, i.e., a multi-compartment device, or dyeing kit, in which a first compartment contains the dyeing composition of the invention and a second compartment contains the oxidizing composition. This device may be equipped with a means for delivering the desired mixture to the hair, such as the devices described in French Patent No. FR 2 586 913.

Other than in the operating examples, or where otherwise indicated, all numbers expressing quantities of ingredients, reaction conditions, and so forth in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Also, where the term “between” is used, the ranges defined include the stated endpoints. Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement.

The examples that follow serve to illustrate the invention without, however, being limiting in nature.

### EXAMPLES

### 1 Synthesis of Compounds

Synthesis of Compound 2

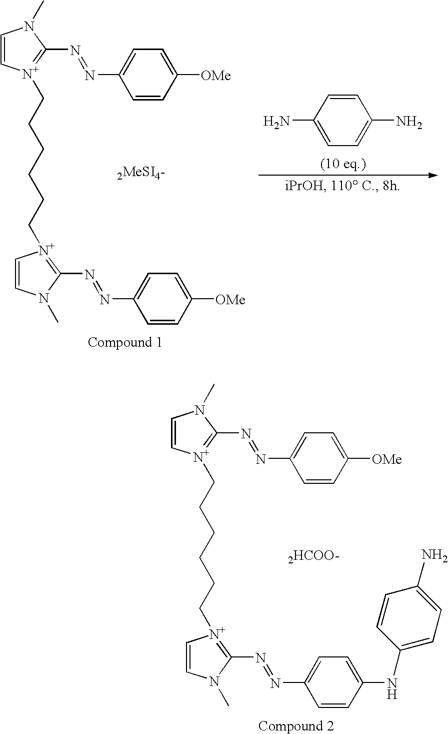

Compound 1 (1.477 g, n=0.002 mol) was reacted in the presence of 1.08 g (n=0.02 mol) of p-phenylenediamine in 20 ml of isopropanol at 110° C. for 8 hours. Concentration of the reaction mixture gave a violet residue. Purification was required by liquid chromatography to give the pure compound 2 in the form of a dark purple-red powder.

The ¹H NMR and mass analyses were in accordance with the expected product.
Synthesis of Compound 6

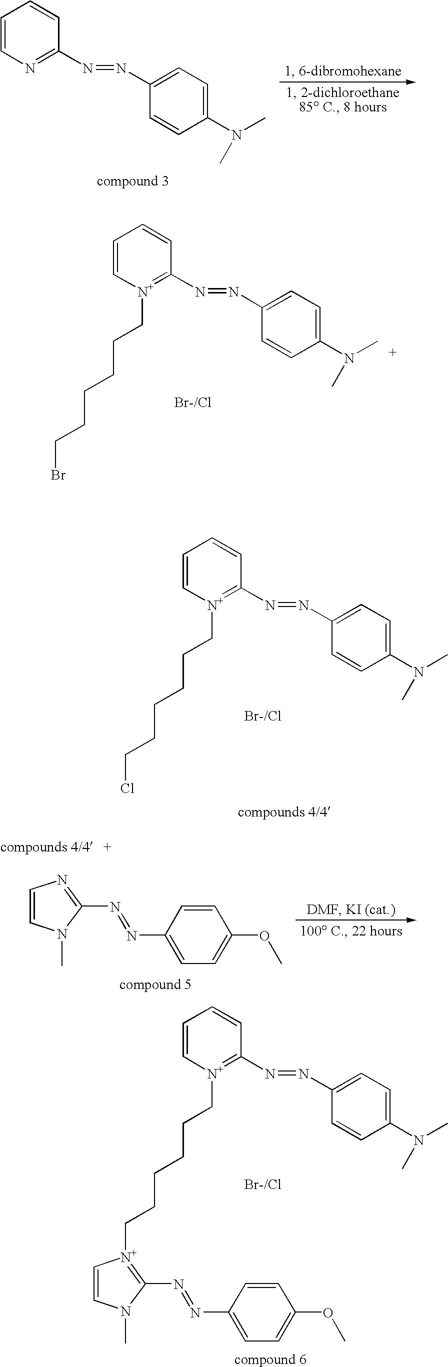

Compound 3 is available commercially (Interchim).

Compound 5 was synthesized in 3 steps:
Coupling reaction of the diazonium salt of p-hydroxyaniline to imidazole.
O-alkylation reaction in the presence of methanol and sulphuric acid.
N-alkylation reaction in the presence of dimethyl carbonate.
   Step 1

1.13 g of compound 3 were stirred at 85° C. for 8 hours in the presence of 3.86 ml of 1,6-dibromohexane in 80 ml of 1,2-dichloroethane. The reaction mixture was subsequently brought to ambient temperature. The reaction mixture was diluted with dichloromethane and washed with water. The organic phase was separated off, dried over sodium sulphate, filtered and concentrated under reduced pressure. This gave a dark violet powder (compound 4/4′), mixture of bromo compound and chloro compound).

The ¹H NMR and mass analyses were in accordance with the expected product (mixture of bromo compound and chloro compound).

Step 2

4.38 g of compound (4/4′) were stirred at 100° C. for 22 hours in the presence of 1.9 g of compound 5 and 0.05 g of Kl in 15 ml of dimethylformamide. The reaction mixture was subsequently brought to ambient temperature and then poured into a mixture containing ethyl acetate and diisopropyl ether (proportion 3/7). A precipitate was formed. It was isolated by filtration. The solid obtained was dissolved in dichloromethane and the solution was then poured into a solution containing ethyl acetate and isopropanol (proportion 4/1). A further precipitate appeared. It was isolated by filtration and dried under vacuum. This gave a dark purple powder corresponding to the expected compound 6.

The ¹H NMR and mass analyses were in accordance with the structure of the expected compound 6.
Synthesis of Compound 7

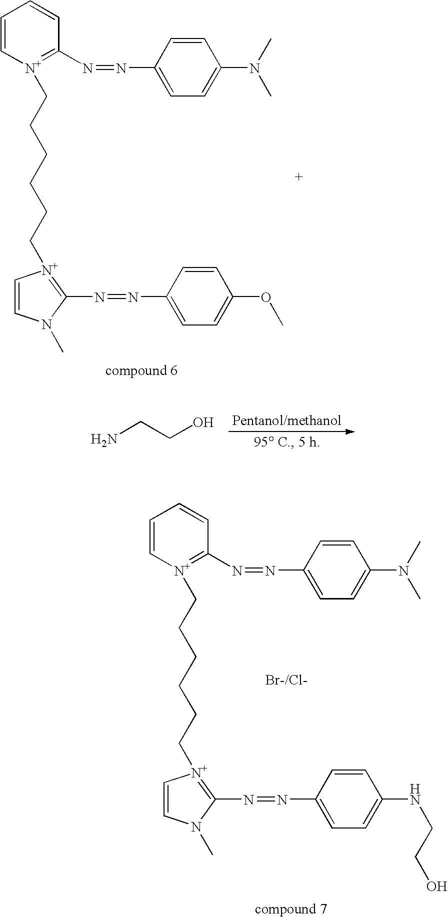

1 g of compound 6 and 0.87 ml of ethanolamine were reacted in 6 ml of pentanol and 1 ml of methanol at 95° C. for 5 hours. The reaction mixture was subsequently brought to ambient temperature and then poured into a mixture containing ethyl acetate and diisopropyl ether. A precipitate was formed. It was isolated by filtration and dried under vacuum. Purification by liquid chromatography gave a dark purple-red powder.

The ¹H NMR and mass analyses were in accordance with the structure of the expected compound 7.
Synthesis of Compound 8

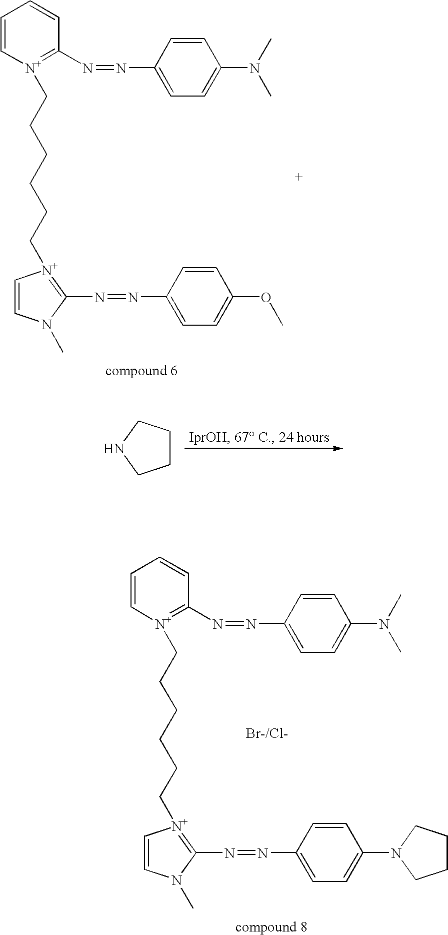

0.68 g of compound 6 and 0.37 ml of pyrrolidine were reacted in 8 ml of isopropanol at 67° C. for 24 hours. The reaction mixture was subsequently brought to ambient temperature and then poured into a mixture containing ethyl acetate and diisopropyl ether. A precipitate was formed. It was isolated by filtration and dried under vacuum. Purification by liquid chromatography gave a fuchsia-colored powder.

The ¹H NMR and mass analyses were in accordance with the structure of the expected compound 8.
Synthesis of Compound 11:

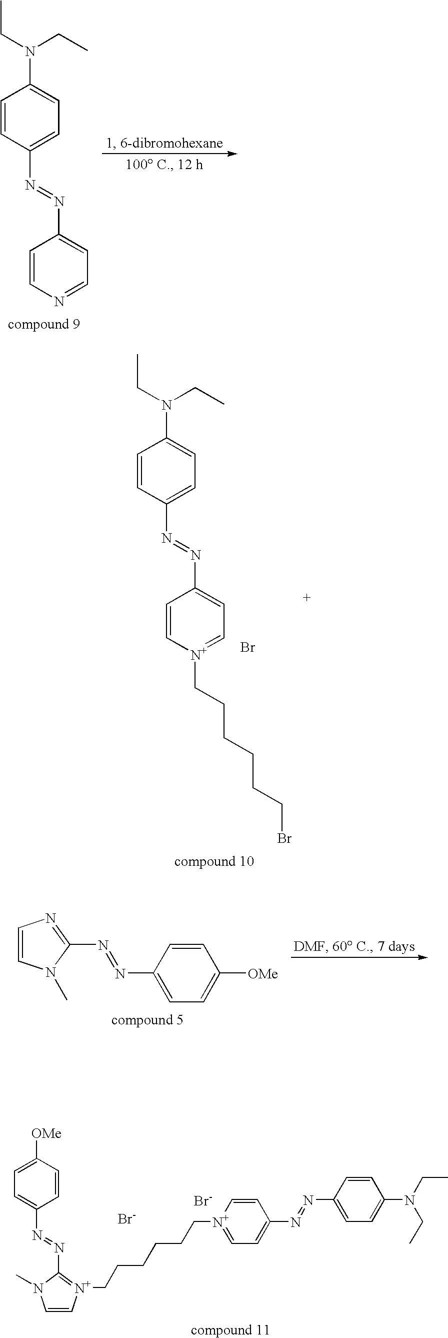

Compound 9 is commercial (Interchim).

Step 1:

Compound 9 (2.5 g) was reacted in the presence of 50 ml of 1,6-dibromohexane at 100° C. for 12 hours. The reaction mixture was brought to ambient temperature beforehand, water was added, and the product was then extracted with dichloromethane. Drying over sodium sulphate, filtration and evaporation gave a violet powder (2 g) corresponding to the compound of structure 10.

The ¹H NMR and mass analyses were in accordance with the expected product.

Step 2:

Compound 10 (0.1 g) and 0.042 g of compound 5 were reacted in the presence of 1 ml of DMF at 60° C. for 7 days. The reaction mixture was brought to ambient temperature beforehand. The residue obtained by precipitation of the reaction mixture from diisopropyl ether was purified by liquid chromatography. A fuchsia powder corresponding to the compound of structure 11 was obtained.

The ¹H NMR and mass analyses were in accordance with the expected product.
Synthesis of Compound 14:

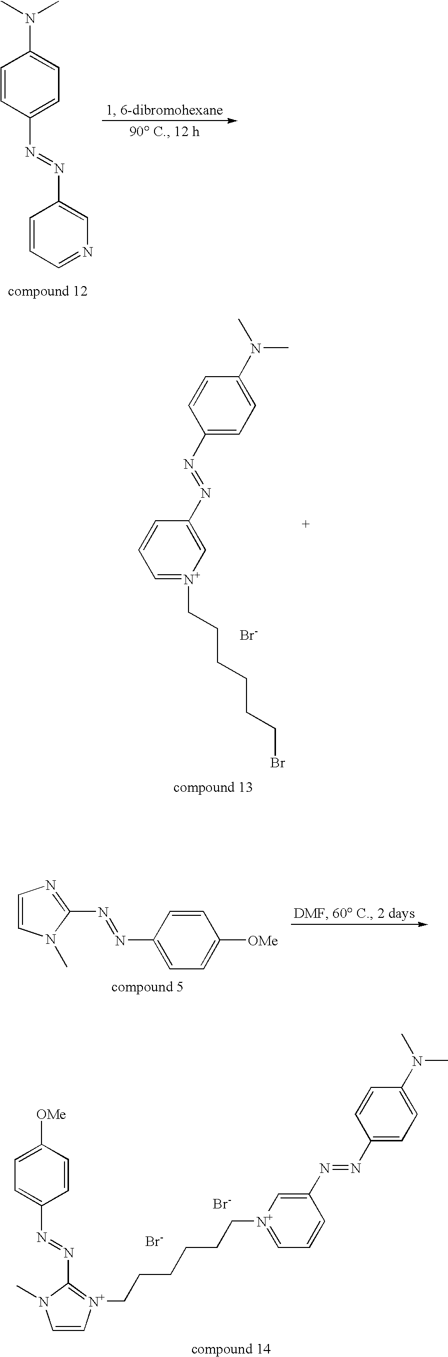

Compound 12 was obtained by reacting the diazonium salt of 3-aminopyridine with dimethylaniline.

Step 1:

Compound 12 (1 g) was reacted in the presence of 6.82 ml of 1,6-dibromohexane at 90° C. for 12 hours. The reaction mixture was brought to ambient temperature beforehand and then poured into a solution containing diisopropyl ether. A precipitate was obtained and was then isolated by filtration and dried. This gave a dark orange-colored powder (2 g) corresponding to the compound of expected structure 13. The ¹H NMR and mass analyses were in accordance with the expected product.

Step 2:

Compound 13 (1 g) and 0.5 g of compound 5 were reacted in the presence of 10 ml of DMF at 90° C. for 2 days. The reaction mixture was brought to ambient temperature beforehand. The residue obtained by precipitating the reaction mixture from diisopropyl ether was purified by liquid chromatography. An orange-yellow powder corresponding to the compound of structure 14 was obtained.

The ¹H NMR and mass analyses were in accordance with the expected product.
Synthesis of Compound 19: 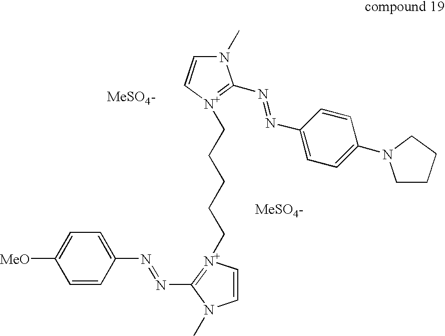
Step 1:

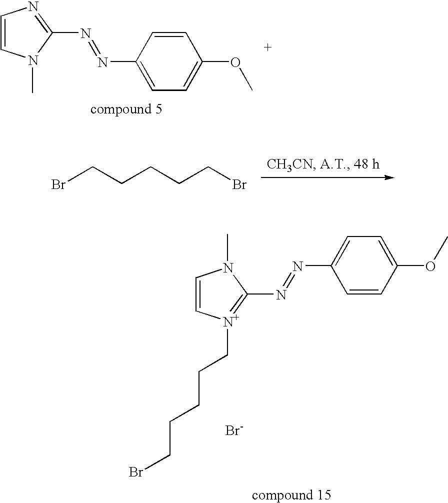

In a three-necked flask with a top-mounted condenser, compound 5 (0.216 g, 1 mmol) was stirred at ambient temperature for 48 hours in the presence of 2 ml of 1,5-dibromopentane in 5 ml of acetonitrile. Following reaction, the reaction mixture was concentrated. The residue obtained was purified by flash silica chromatography. 208 mg of an orange-yellow powder corresponding to compound 15 were obtained.

The analyses were in accordance with the expected product.
Step 2:

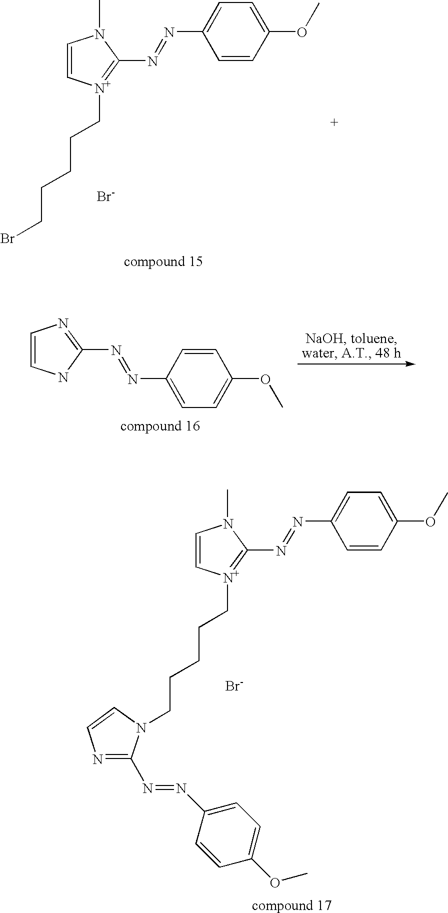

Compound 16 was obtained by reacting the diazonium salt of p-anisidine with imidazole.

In a three-necked flask with a top-mounted condenser, compound 15 (0.223 g, 0.5 mmol), compound 16 (0.121 mg, 0.6 mmol) and sodium hydroxide (0.025 g; 0.625 mmol) were stirred at ambient temperature for 48 hours in the presence of 50 ml of water and 4 ml of toluene.

After reaction, the reaction mixture was concentrated. The residue obtained was washed a number of times with toluene. 240 mg of an orange-yellow powder corresponding to compound 17 were obtained.

The analyses were in accordance with the expected product.
Step 3:

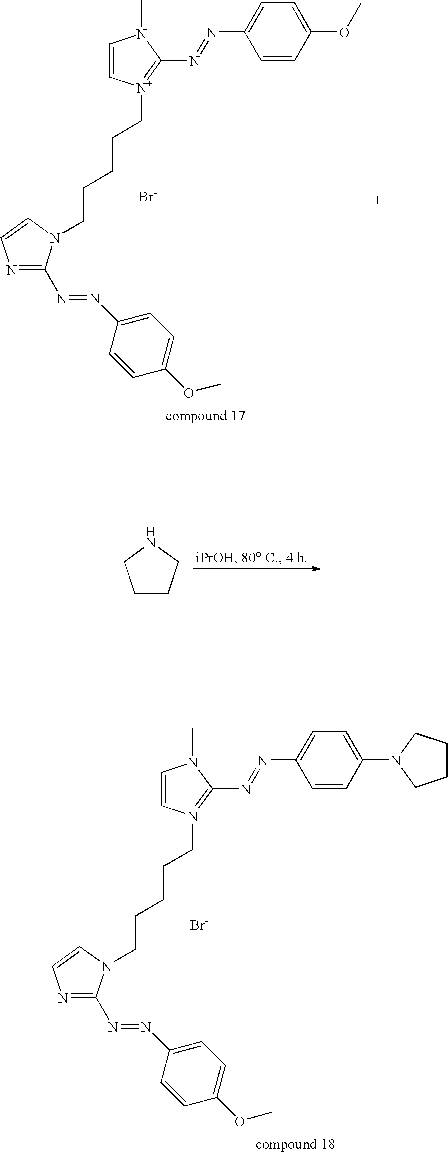

In a three-necked flask with a top-mounted condenser, compound 17 (0.283 g, 0.5 mmol) and pyrrolidine (0.043 g; 0.6 mmol) were stirred at 80° C. for 4 hours in the presence of 10 ml of isopropanol.

After reaction, the reaction mixture was concentrated. The residue obtained was purified by flash silica chromatography. 113 mg of a fuchsia-red powder corresponding to compound 18 were obtained.

The analyses were in accordance with the expected product.
Step 4:

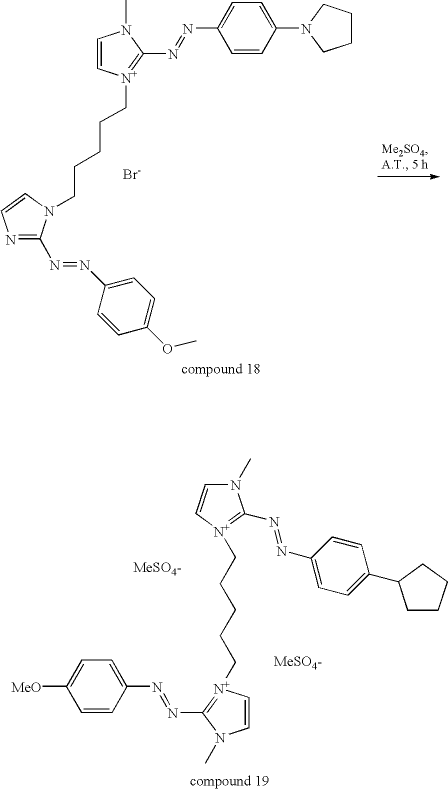

In a three-necked flask with a top-mounted condenser, compound 18 (0.121 g, 0.2 mmol) was stirred at ambient temperature for 5 hours in the presence of dimethyl sulphate (55 mg; 0.44 mmol) in 5 ml of 1,2-dichloroethane.

After reaction, the reaction mixture was concentrated. The residue obtained was washed with 5 ml of ethyl acetate. After the resulting precipitate had been isolated by filtration and dried, 113 mg of a fuchsia red powder corresponding to compound 19 were obtained.

The analyses were in accordance with the expected product.
Synthesis of Compound 20:

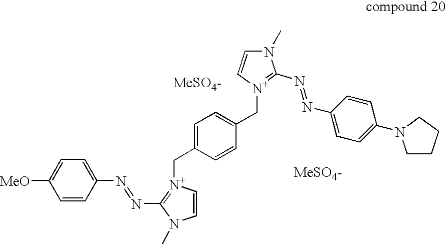

The same methodology was applied to give compound 20. This was done using the following halo derivative:

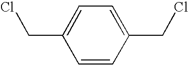

2.5 g of a fuchsia red powder corresponding to compound 20 were obtained.
Synthesis of Compound 21:

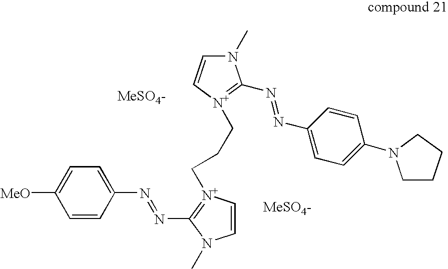

The same methodology was applied to give compound 21. This was done using the following halo derivative:

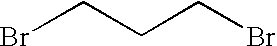

1.5 g of a fuchsia red powder corresponding to compound 21 were obtained.

### 2

### Example of Dyeing Under Non-Oxidizing Conditions

5×10⁻⁴ mol of compound 6, obtained above, were dissolved in 5 ml of a mixture of water (2.5 ml) and pH 10 buffer (2.5 ml) with the following composition:

2 g of ammonium acetate

40 ml of water

NH₃ at 20% to a pH of 9-10 water to 100 ml.

100 g of the above composition were applied to hair at ambient temperature for 30 minutes. The hair was then rinsed with water and dried.

The hair was colored claret.

What is claimed is:

1. A dissymmetrical cationic diazo compound chosen from those of formulae (I), (II), (III) and (IV), their resonance forms, their acid addition salts, and their solvates, wherein in the compounds of formulae (I), (II), (III) and (IV), the formula members attached to each side of the linker L are either compositionally different or positionally different such that the compounds as a whole are dissymmetrical: 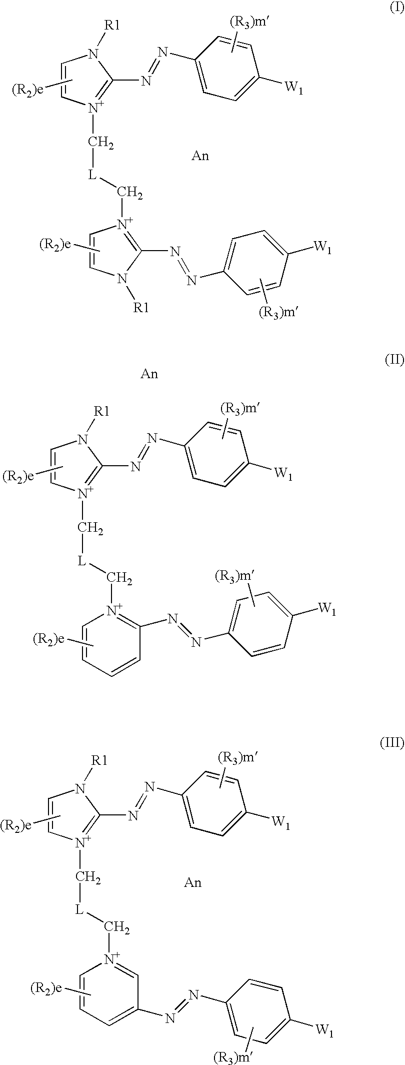 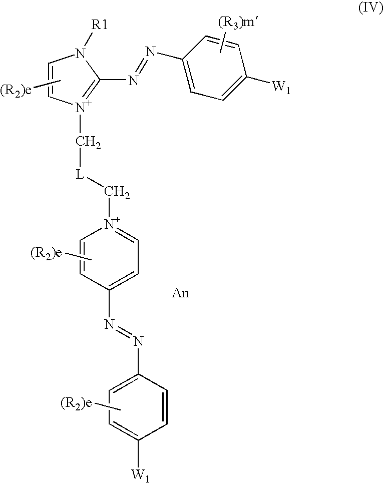
wherein:
the radicals R₁, which are identical or different, independently of one another are chosen from:
optionally substituted C₁-C₄ alkyl radicals;
optionally substituted phenyl radicals;
optionally substituted benzyl radicals;
the radicals R₂, which are identical or different, independently of one another are chosen from:
optionally substituted C₁-C₁₆ alkyl radicals optionally interrupted by at least one heteroatom and/or at least one group containing at least one heteroatom, said alkyl radical being further optionally substituted by at least one group chosen from thio (—SH), C₁-C₄ thioalkyl; C₁-C₄ alkylsulphinyl and C₁-C₄ alkylsulphonyl groups;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group, wherein the two alkyl radicals may optionally form, with the nitrogen atom to which they are attached, a heterocycle containing 1 to 3 heteroatoms chosen from N, O and S, and containing 5 to 7 ring members, wherein said heterocycle is saturated or unsaturated, aromatic or non-aromatic and is optionally substituted;
alkylcarbonylamino groups (RCO—NR′—) wherein R is a C₁-C₄ alkyl radical and R′ is chosen from hydrogen and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein R is a C₁-C₄ alkyl radical and R′ is chosen from a hydrogen atom and a C₁-C₄ alkyl radical;
optionally substituted aryl radicals;
optionally substituted (C₁-C₄)alkylaryl radicals;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
thio groups (HS—);
alkylthio groups (RS—) wherein R is an optionally substituted C₁-C₄ alkyl radical;
when e is 2, for each of the formulae (I), (II), (III) and (IV), two radicals R₂ may optionally form, with the carbon atoms to which they are attached, a secondary ring, aromatic or non-aromatic, containing 5 or 6 ring members, which is optionally substituted by at least one identical or different group chosen from hydroxyl, C₁-C₄ alkyl, C₁-C₄ alkoxy, C₂-C₄ (poly)hydroxyalkoxy, amino, and amino substituted by one or two identical or different C₁-C₄ alkyl radicals which optionally carry at least one hydroxyl group;
e is an integer ranging from 0 to 4; when e is less than 4, the unsubstituted carbon atom(s) of the heterocycle of the formulae (I), (II), (III) and (IV) carry a hydrogen atom;
the radicals R₃, from formulae (I), (II), (III) and (IV), which are identical or different, independently of one another are chosen from:
optionally substituted C₁-C₁₆ alkyl radicals optionally interrupted by at least one heteroatom or by at least one group containing at least one heteroatom;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group; wherein the two alkyl radicals may optionally form, with the nitrogen atom to which they are attached, a heterocycle containing 1 to 3 heteroatoms chosen from N, O and S, containing 5 to 7 ring members, and which is saturated or unsaturated, aromatic or non-aromatic and is optionally substituted;
alkylcarbonylamino groups (RCO—NR′—) wherein the radical R is a C₁-C₄ alkyl radical and the radical R′ is chosen from a hydrogen atom and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein radicals R and R′ independently of one another are chosen from a hydrogen atom and a C₁-C₄ alkyl radical;
a thio group (HS—);
alkylthio groups (RS—) wherein R is a C₁-C₄ alkyl radical;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
when m′ is greater than or equal to 2, two adjacent radicals R₃ may form, with the carbon atoms to which they are attached, a secondary ring, aromatic or non-aromatic containing 6 ring members, which is optionally substituted by at least one identical or different group chosen from: hydroxyl, C₁-C₄ alkyl, C₁-C₄ alkoxy, C₂-C₄ (poly)hydroxyalkoxy, C₁-C₄ alkylcarbonylamino, amino, and amino substituted by one or two identical or different C₁-C₄ alkyl radicals which optionally carry at least one hydroxyl group;
m′ is an integer from 0 to 4; when m′ is less than 4, then the unsubstituted carbon atom(s) of the aromatic ring of the formulae (I), (II), (III) and (IV) carry a hydrogen atom;
the W₁ groups of the formulae (I), (II), (III) and (IV), which are identical or different, independently of one another, are chosen from:
hydrogen atoms;
halogen atoms chosen from bromine, chlorine and fluorine;
a group chosen from —NR₅R₆, OR₇, —NR₄-Ph-NR₅R₆, —NR₄-Ph-OR₇, —O-Ph-OR₇ and —O-Ph-NR₅R₆ groups, wherein:
R₄ and R₇, which are identical or different, are chosen from hydrogen atoms, optionally substituted C₁-C₂₀ alkyl radicals, optionally substituted C₁-C₃ aralkyl radicals and optionally substituted phenyl radicals;
R₅ and R₆, which are identical or different, are chosen from hydrogen atoms, optionally substituted C₁-C₂₀ alkyl radicals, optionally substituted phenyl radicals, optionally substituted C₁-C₃ aralkyl radicals and alkylcarbonyl radicals (R—CO—) wherein R is a C₁-C₄ alkyl radical;
R₅ and R₆ may optionally form, with the nitrogen atom to which they are attached, a heterocycle containing 1 to 3 heteroatoms, chosen from N, O and S, and containing 5 to 7 ring members, which is saturated or unsaturated, aromatic or non-aromatic and is optionally substituted;
R₅ and R₆ may optionally form, with the carbon atom of the aromatic ring adjacent to that to which —NR₅R₆ is attached, a 5- or 6-membered saturated heterocycle;
Ph is an optionally substituted phenyl radical;
L is chosen from a cationic linker and a non-cationic linker;
An is chosen from at least one identical or different cosmetically acceptable anions which ensure the electroneutrality of the compounds of formula (I), (II), (III) and (IV).

2. The compound according to claim 1, wherein when R₂ and/or R₃ is chosen from optionally substituted C₁-C₁₆ alkyl radicals optionally interrupted by at least one heteroatom and/or at least one group containing at least one heteroatom, said heteroatoms and said groups are chosen from oxygen, nitrogen, sulphur, —CO—, SO₂—, and combinations thereof.

3. The compound according to claim 1, wherein said radicals R₁ are chosen from:
C₁-C₄ alkyl radicals optionally substituted by at least one radical chosen from hydroxyl, C₁-C₂ alkoxy, amino and C₁-C₂ (di)alkylamino radicals; and
optionally substituted benzyl radicals.

4. The compound according to claim 1, wherein said radicals R₂, which are identical or different, are chosen from:
halogen atoms chosen from chlorine and fluorine;
C₁-C₄ alkyl radicals optionally substituted by at least one identical or different radical chosen from hydroxyl, C₁-C₂ alkoxy, C₂-C₄ (poly)hydroxyalkoxy, amino, C₁-C₂ (di)alkylamino, thio (—SH), C₁-C₄ alkylsulphinyl, C₁-C₄ alkylsulphonyl and C₁-C₄ thioalkyl radicals;
phenyl radicals optionally substituted by at least one identical or different radical chosen from hydroxyl, C₁-C₂ alkoxy, C₂-C₄ (poly)hydroxyalkoxy, amino and C₁-C₂ (di)alkylamino radicals and a halogen atom;
C₁-C₄ alkoxy radicals;
C₁-C₄ alkylsulphonylamino radicals;
C₂-C₄ (poly)hydroxyalkoxy radicals;
amino radicals;
C₁-C₂ (di)alkylamino radicals;
C₂-C₄ (poly)hydroxyalkylamino radicals;
alkylsulphonylamino radicals (RSO₂N—) wherein R is a C₁-C₄ alkyl radical;
aminosulphonyl radicals ((R)₂NSO₂—) wherein said radicals R independently of one another are chosen from a hydrogen atom and a C₁-C₄ alkyl radical;
alkylthio radicals (RS—) wherein said radical R is a C₁-C₄ alkyl radical;
alkylsulphinyl radicals (RSO—) wherein said radical R is a C₁-C₄ alkyl radical;
alkylsulphonyl radicals (R—SO₂—) wherein said radical R is a C₁-C₄ alkyl radical;
alkylcarbonylamino radicals (RCONR′—) wherein said radical R is chosen from a hydrogen atom and a C₁-C₄ alkyl radical and radical R′ is chosen from a hydrogen atom and a C₁-C₄ alkyl radical.

5. The compound according to claim 1, wherein the two radicals R₂ from formulae (I), (II), (III) and (IV) may optionally form, with the carbon atoms to which they are attached, a secondary, 6-membered aromatic ring optionally substituted by at least one identical or different group chosen from hydroxyl, C₁-C₄ alkyl, C₁-C₄ alkoxy, amino, and amino substituted by one or two identical or different C₁-C₄ alkyl radicals which optionally carry at least one hydroxyl or methylcarbonylamino group.

6. The compound according to claim 1, wherein the radicals R₃, which are identical or different, are chosen from:
optionally substituted C₁-C₁₆ alkyl radicals;
halogen atoms;
hydroxyl groups;
C₁-C₂ alkoxy radicals;
C₂-C₄ (poly)hydroxyalkoxy radicals;
amino radicals;
amino radicals substituted by one or two identical or different C₁-C₄ alkyl radicals which optionally carry at least one identical or different group chosen from hydroxyl and C₁-C₄ alkoxy, wherein the two alkyl radicals may optionally form, with the nitrogen atom to which they are attached a heterocycle containing 1 to 3 heteroatoms chosen from N, O and S, the heterocycle containing 5 to 7 ring members, being saturated or unsaturated, aromatic or non-aromatic, and being optionally substituted;
alkylcarbonylamino radicals (RCO—NR′—) wherein the radical R is a C₁-C₄ alkyl radical and the radical R′ is chosen from a hydrogen and a C₁-C₄ alkyl radical;
alkylsulphonylamino radicals (R′SO₂—NR—) wherein the radical R is chosen from a hydrogen atom and a C₁-C₄ alkyl radical and the radical R′ is a C₁-C₄ alkyl radical;
aminosulphonyl radicals ((R)₂N—SO₂—) wherein the radicals R′, which are identical or different, are chosen from a hydrogen atom and a C₁-C₄ alkyl radical;
alkylthio radicals (RS—) wherein the radical R is a C₁-C₄ alkyl radical; and
alkylsulphonyl radicals (R—SO₂—) wherein the radical R is a C₁-C₄ alkyl radical.

7. The compound according to claim 1, wherein the radicals R₃, which are identical or different are chosen from:
C₁-C₄ alkyl radicals which are optionally substituted by at least one identical or different radical chosen from hydroxyl radicals C₁-C₂ alkylcarbonylamino radicals, amino radicals substituted by two identical or different C₁-C₂ alkyl radicals which optionally carry at least one identical or different group chosen from hydroxyl or C₁-C₂ alkoxy, wherein the two alkyl radicals may optionally form, with the nitrogen atom to which they are attached a 5- or 6-membered heterocycle which is saturated or unsaturated and is optionally aromatic;
C₂-C₄ hydroxyalkoxy radicals;
halogen atoms chosen from chlorine and fluorine;
amino radicals;
amino radicals substituted by one or two identical or different C₁-C₂ alkyl radicals which optionally carry at least one hydroxyl group;
methylcarbonylamino radicals;
methylsulphonylamino radicals;
hydroxyl radicals;
C₁-C₂ alkoxy radicals; and
methylsulphonyl radicals.

8. The compound according to claim 7, wherein when R₃ is chosen from C₁-C₄ alkyl radicals substituted by amino radicals substituted by two identical or different C₁-C₂ alkyl radicals and wherein when the two alkyl radicals optionally form, with the nitrogen atom to which they are attached, a 5- or 6-membered saturated or unsaturated and optionally aromatic heterocycle, said heterocycle is chosen from pyrrolidine, piperazine, homopiperazine, pyrrole, imidazole and pyrazole.

9. The compound according to claim 1, wherein, when the coefficient m′ is greater than or equal to 2, two adjacent radicals R₃ may form, with the carbon atoms to which they are attached, a secondary, 6-membered aromatic ring optionally substituted by at least one identical or different group chosen from hydroxyl groups, —NR₄—Ph, —NR₄-Ph-NR₅R₆ and —NR₄-Ph-OR₇ groups, C₁-C₄ alkyl groups, C₁-C₄ alkoxy groups, C₂-C₄ (poly)hydroxyalkoxy groups, C₁-C₄ alkylcarbonylamino groups, amino groups, and amino groups substituted by one or two identical or different C₁-C₄ alkyl radicals which optionally carry at least one hydroxyl group.

10. The compound according to claim 9, wherein two adjacent radicals R₃ may form, with the carbon atoms to which they are attached, a secondary, 6-membered aromatic ring which is optionally substituted by at least one identical or different group chosen from hydroxyl, methoxy, ethoxy, 2-hydroxyethyloxy, amino, methylcarbonylamino, (di)-2-hydroxyethylamino, —NH-Ph, —NH-Ph-NH₂, —NH-Ph-NHCOCH₃, —NH-Ph-OH and —NH-Ph-OCH₃ groups.

11. The compound according to claim 10, wherein R₄ and R₇ independently of one another are chosen from:
hydrogen atoms;
C₁-C₆ alkyl radicals which are optionally substituted by at least one identical or different group; and
aryl and arylalkyl radicals, the aryl moiety being optionally substituted by at least one identical or different group.

12. The compound according to claim 11, wherein when R₄ and R₇ independently of one another are chosen from aryl and arylalkyl radicals, the aryl moiety being optionally substituted by at least one identical or different group chosen from chlorine atoms, amino groups, hydroxyl groups, C₁-C₂ alkoxy groups and amino groups which are mono- or disubstituted by two identical or different C₁-C₄ alkyl radicals which optionally carry at least one hydroxyl group.

13. The compound according to claim 11, wherein when R₄ and R₇ independently of one another are chosen from C₁-C₆ alkyl radicals, the alkyl radicals may be optionally substituted by at least one identical or different group chosen from hydroxyl and C₁-C₂ alkoxy.

14. The compound according to claim 1, wherein the radicals R₅ and R₆, which are identical or different, are chosen from:
hydrogen atoms;
alkylcarbonyl radicals (R—CO—) wherein R is an optionally substituted C₁-C₄ alkyl radical;
a C₁-C₆ alkyl radical which is optionally substituted by at least one identical or non-identical group chosen from hydroxyl, C₁-C₂ alkoxy, amino, C₁-C₄ (di)alkylamino; the alkyl radical may further be substituted by at least one identical or different group chosen from C₁-C₄ alkylsulphonyl, C₁-C₄ alkylsulphinyl and C₁-C₄ alkylcarbonyl;
aryl and arylalkyl radical, the aryl moiety being optionally substituted by at least one identical or different radical.

15. The compound according to claim 14, wherein when the radicals R₅ and
R₆, which are identical or different, are chosen from aryl and arylalkyl radicals, the aryl moiety may be optionally substituted by at least one identical or different radical chosen from a chlorine atom, an amino group, a hydroxyl group, a C₁-C₄ alkoxy group and an amino group which is mono- or disubstituted by two identical or different C₁-C₄ alkyl radicals which optionally carry at least one hydroxyl group.

16. The compound according to claim 13, wherein the radicals R₅ and R₆, which are identical or different, are chosen from:
hydrogen atoms;
methylcarbonyl, ethylcarbonyl and propylcarbonyl radicals;
optionally substituted C₁-C₃ alkyl radicals;
phenyl radicals which are optionally substituted by at least one identical or different radical chosen from hydroxyl radicals, C₁-C₂ alkoxy radicals, amino radicals, amino radicals substituted by at least one identical or different C₁-C₄ alkyl group which optionally carries at least one hydroxyl group.

17. The compound according to claim 16, wherein when the radicals R₅ and R₆, which are identical or different, are chosen from optionally substituted C₁-C₃ alkyl radicals, said radicals are chosen from methyl, ethyl, 2-hydroxyethyl and 2-methoxyethyl radicals.

18. The compound according to claim 1, wherein the radicals R₅ and R₆ form, together with the nitrogen atom to which each is attached, a heterocycle containing 1 to 3 heteroatoms chosen from N, O and S, and containing 5 to 7 ring members, which is saturated or unsaturated, aromatic or non-aromatic, and is optionally substituted.

19. The compound according to claim 18, wherein the heterocycle containing 5 to 7 ring members is chosen from: piperidine, 2-(2-hydroxyethylpiperidine), 4-(aminomethyl)piperidine, 4-(2-hydroxyethyl)piperidine, 4-(dimethylamino)piperidine, piperazine, 1-methylpiperazine,1-(2-hydroxyethyl)piperazine, 1-(2-aminoethyl)piperazine, 1-hydroxyethylethoxypiperazine, homopiperazine, 1-methyl-1,4-perhydrodiazepine, pyrrole, 1,4-dimethylpyrrole, 1-methyl-4-ethylpyrrole, and 1-methyl-4-propylpyrrole.

20. The compound according to claim 1, wherein the radicals R₅ and R₆ may form, with the carbon atom of the aromatic ring optionally substituted by a hydroxyl and adjacent to that to which —NR₅R₆ is attached, a 5- or 6-membered saturated heterocycle.

21. The compound according to claim 1, wherein L is a non-cationic linker chosen from:
a covalent bond;
optionally substituted C₁-C₄₀ alkyl radicals optionally interrupted by a saturated or unsaturated, aromatic or non-aromatic (hetero)cycle containing 3 to 7 ring members which are optionally substituted and optionally fused, said alkyl radical being optionally interrupted by at least one heteroatom or by at least one group containing at least one heteroatom, the linker L not containing an azo, nitro, nitroso or peroxo bond; and
optionally substituted phenyl radicals.

22. The compound according to claim 21, wherein when L is chosen from an optionally substituted C₁-C₄₀ alkyl radical optionally interrupted by at least one heteroatom or by at least one group containing at least one heteroatom, said heteroatoms and groups are chosen from oxygen, nitrogen, sulphur, —CO—, SO₂—, and combinations thereof.

23. The compound according to claim 1, wherein L is a cationic linker chosen from:
C₂-C₄₀ alkyl radicals which carry at least one cationic charge and are optionally substituted and/or optionally interrupted by at least one saturated or unsaturated, aromatic or non-aromatic, identical or different (hetero)cycle containing 3 to 7 ring members and/or optionally interrupted by at least one heteroatom or group containing at least one heteroatom, or combinations thereof, the linker L containing no azo, nitro, nitroso or peroxo bond; and with the proviso that the linker L carries at least one cationic charge.

24. The compound according to claim 23, wherein said heteroatoms and groups containing at least one heteroatom are chosen from oxygen, nitrogen, sulphur, —CO—, —SO₂— and combinations thereof.

25. The compound according to claim 1, wherein An is an organic anion, an inorganic anion and/or an anion mixture allowing the charge or charges on the compounds of formula (I), (II), (III) and (IV) to be balanced, and wherein An is chosen from a halide; a hydroxide; a sulphate; a hydrogensulphate; alkylsulphates wherein the linear or branched alkyl moiety is C₁-C₆; carbonates and hydrogencarbonates; salts of carboxylic acids; alkylsulphonates wherein the linear or branched alkyl moiety is C₁-C₆; arylsulphonates wherein the aryl moiety is optionally substituted by at least one C₁-C₄ alkyl radical; and alkylsulphonyls.

26. The compound according to claim 25, wherein in the definition of An,
the halides are chosen from chloride, bromide, fluoride, and iodide,
the alkylsulphates wherein the linear or branched alkyl moiety is C₁-C₆ are chosen from a methylsulphate ion and an ethylsulphate ion;
the salts of carboxylic acids are chosen from formate, acetate, citrate, tartrate, and oxalate;
the alkylsulphonates wherein the linear or branched alkyl moiety is C₁-C₆ are methylsulphonate ions;
the aryl moiety of the arylsulphonates is phenyl and is optionally substituted by at least one C₁-C₄ alkyl radical chosen from 4-tolylsulphonate; and/or the alkylsulphonyls are chosen from mesylate.

27. The compound according to claim 17 wherein the compound is chosen from those of formulae (I′), (I″), (I′″), (II′), (II″), (II′″), (III′), (III″), (III′″), (IV′), (IV″), (IV′″) below, their resonance forms, their acid addition salts and their solvates: 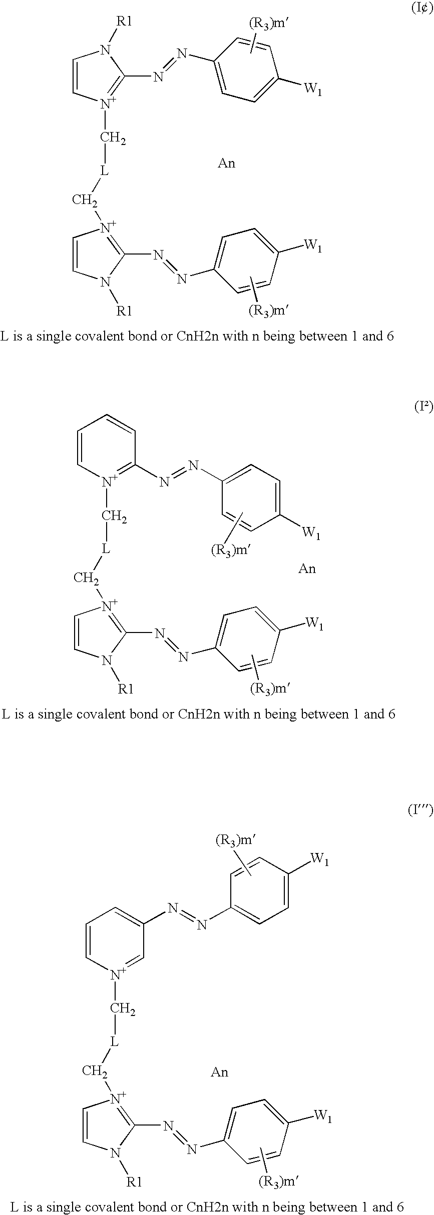 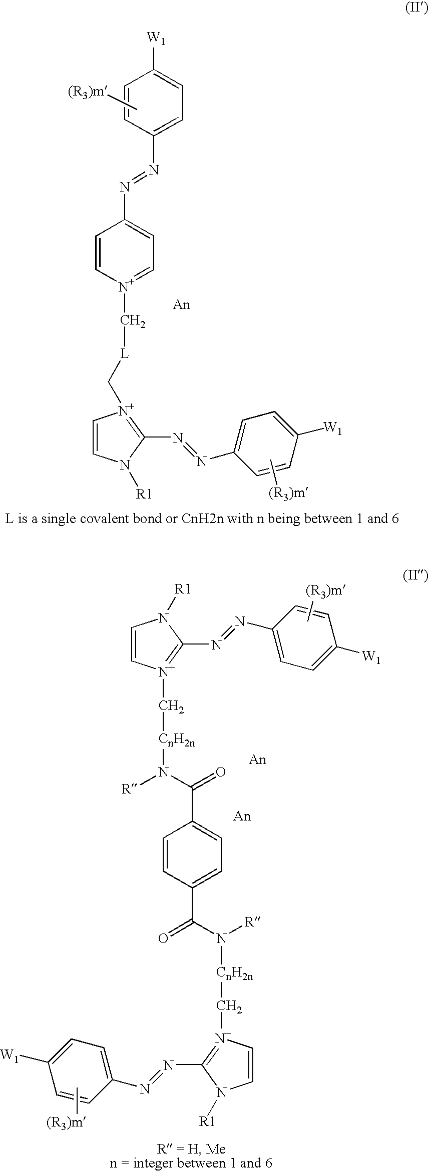 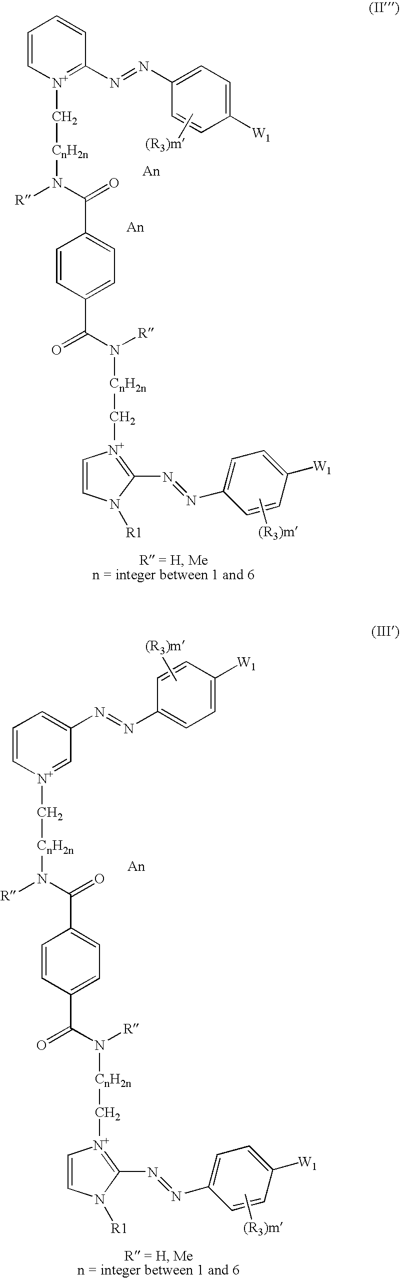 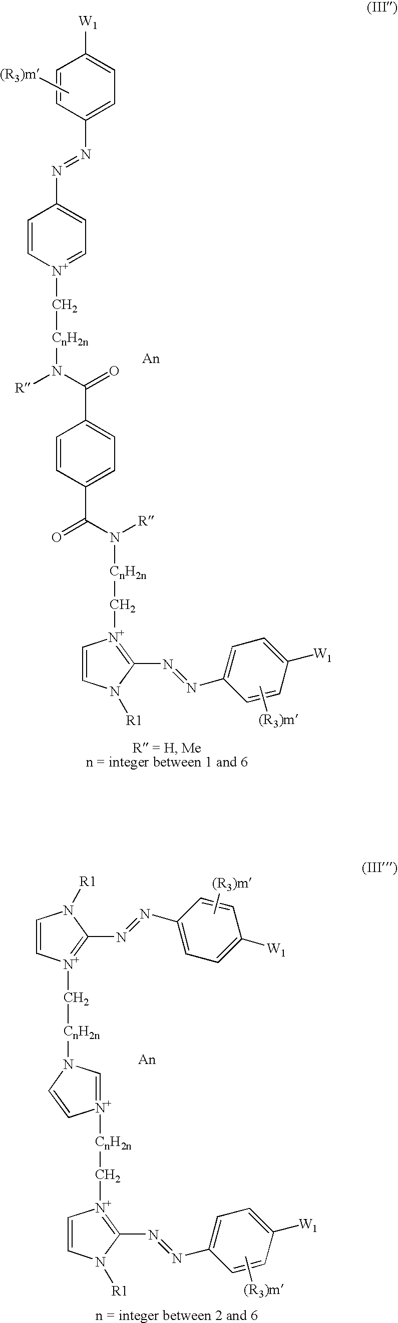 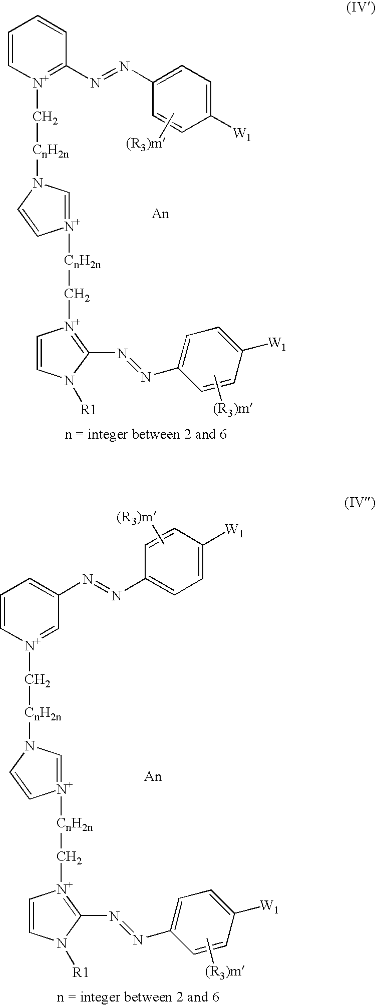 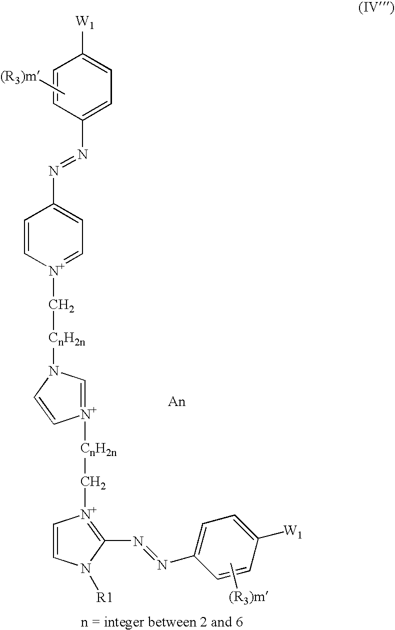
wherein R₁, R₃, W₁ and m′ are as defined in claim 1.

28. A dyeing composition comprising, in a medium appropriate for dyeing keratin fibers, a direct dye chosen from at least one compound chosen from those of formulae (I), (II), (III) and (IV), acid addition salts thereof, and solvates thereof, wherein in the compounds of formulae (I), (II), (III) and (IV), the formula members attached to each side of the linker L are either compositionally different or positionally different such that the compounds as a whole are dissymmetrical: 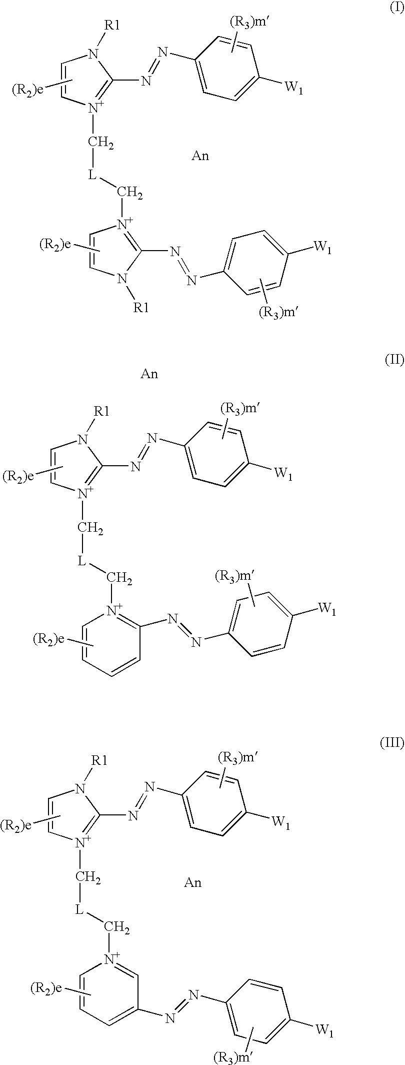 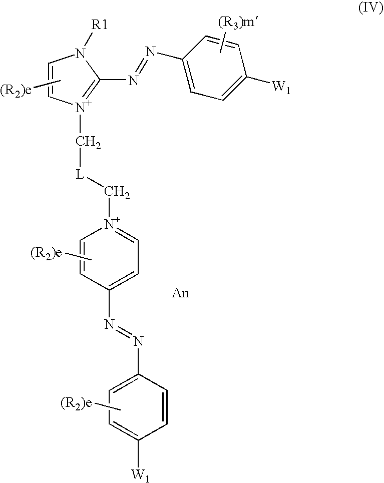
wherein:
the radicals R₁, which are identical or different, independently of one another are chosen from:
optionally substituted C₁-C₄ alkyl radicals;
optionally substituted phenyl radicals;
optionally substituted benzyl radicals;
the radicals R₂, which are identical or different, independently of one another are chosen from:
optionally substituted C₁-C₁₆ alkyl radicals optionally interrupted by at least one heteroatom and/or at least one group containing at least one heteroatom, said alkyl radical being further optionally substituted by at least one group chosen from thio (—SH), C₁-C₄ thioalkyl; C₁-C₄ alkylsulphinyl and C₁-C₄ alkylsulphonyl groups;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group, wherein the two alkyl radicals may optionally form, with the nitrogen atom to which they are attached, a heterocycle containing 1 to 3 heteroatoms chosen from N, O and S, and containing 5 to 7 ring members, wherein said heterocycle is saturated or unsaturated, aromatic or non-aromatic and is optionally substituted;
alkylcarbonylamino groups (RCO—NR′—) wherein R is a C₁-C₄ alkyl radical and R′ is chosen from hydrogen and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein R is a C₁-C₄ alkyl radical and R′ is chosen from a hydrogen atom and a C₁-C₄ alkyl radical;
optionally substituted aryl radicals;
optionally substituted (C₁-C₄)alkylaryl radicals;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
thio groups (HS—);
alkylthio groups (RS—) wherein R is an optionally substituted C₁-C₄ alkyl radical;
when e is 2, for each of the formulae (I), (II), (III) and (IV), two radicals R₂ may optionally form, with the carbon atoms to which they are attached, a secondary ring, aromatic or non-aromatic, containing 5 or 6 ring members, which is optionally substituted by at least one identical or different group chosen from hydroxyl, C₁-C₄ alkyl, C₁-C₄ alkoxy, C₂-C₄ (poly)hydroxyalkoxy, amino, and amino substituted by one or two identical or different C₁-C₄ alkyl radicals which optionally carry at least one hydroxyl group;
e is an integer ranging from 0 to 4; when e is less than 4, the unsubstituted carbon atom(s) of the heterocycle of the formulae (I), (II), (III) and (IV) carry a hydrogen atom, the radicals R₃, from formulae (I), (II), (III) and (IV), which are identical or different, independently of one another are chosen from:
optionally substituted C₁-C₁₆ alkyl radicals optionally interrupted by at least one heteroatom or by at least one group containing at least one heteroatom;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group; wherein the two alkyl radicals may optionally form, with the nitrogen atom to which they are attached, a heterocycle containing 1 to 3 heteroatoms chosen from N, O and S, containing 5 to 7 ring members, and which is saturated or unsaturated, aromatic or non-aromatic and is optionally substituted;
alkylcarbonylamino groups (RCO—NR′—) wherein the radical R is a C₁-C₄ alkyl radical and the radical R′ is chosen from a hydrogen atom and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein radicals R and R′ independently of one another are chosen from a hydrogen atom and a C₁-C₄ alkyl radical;
thio groups (HS—);
alkylthio groups (RS—) wherein R is a C₁-C₄ alkyl radical;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
when m′ is greater than or equal to 2, two adjacent radicals R₃ may form, with the carbon atoms to which they are attached, a secondary ring, aromatic or non-aromatic containing 6 ring members, which is optionally substituted by at least one identical or different group chosen from: hydroxyl, C₁-C₄ alkyl, C₁-C₄ alkoxy, C₂-C₄ (poly)hydroxyalkoxy, C₁-C₄ alkylcarbonylamino, amino, and amino substituted by one or two identical or different C₁-C₄ alkyl radicals which optionally carry at least one hydroxyl group;
m′ is an integer from 0 to 4; when m′ is less than 4, then the unsubstituted carbon atom(s) of the aromatic ring of the formulae (I), (II), (III) and (IV) carry a hydrogen atom;
the W₁ groups of the formulae (I), (II), (III) and (IV), which are identical or different, independently of one another, are chosen from:
hydrogen atoms;
halogen atoms chosen from bromine, chlorine and fluorine;
a group chosen from —NR₅R₆, OR₇, —NR₄-Ph-NR₅R₆, —NR₄-Ph-OR₇, —O-Ph-OR₇ and —O-Ph-NR₅R₆ groups, wherein:
R₄ and R₇, which are identical or different, are chosen from hydrogen atoms, optionally substituted C₁-C₂₀ alkyl radicals, optionally substituted C₁-C₃ aralkyl radicals and optionally substituted phenyl radicals;
R₅ and R₆, which are identical or different, are chosen from hydrogen atoms optionally substituted C₁-C₂₀ alkyl radicals, optionally substituted phenyl radicals, optionally substituted C₁-C₃ aralkyl radicals and alkylcarbonyl radicals (R—CO—) wherein R is a C₁-C₄ alkyl radical;
R₅ and R₆ may optionally form, with the nitrogen atom to which they are attached, a heterocycle containing 1 to 3 heteroatoms, chosen from N, O and S, and containing 5 to 7 ring members, which is saturated or unsaturated, aromatic or non-aromatic and is optionally substituted;
R₅ and R₆ may optionally form, with the carbon atom of the aromatic ring adjacent to that to which —NR₅R₆ is attached, a 5- or 6-membered saturated heterocycle;
Ph is an optionally substituted phenyl radical;
L is chosen from a cationic linker and a non-cationic linker;
An is chosen from at least one identical or different cosmetically acceptable anions which ensure the electroneutrality of the compounds of formula (I), (II), (III) and (IV).

29. The dyeing composition according to claim 28, wherein the at least one compound is present in the composition in an amount ranging from 0.001% to 20% by weight relative to the total weight of the dyeing composition.

30. The dyeing composition according to claim 29, wherein the at least one compound is present in the composition in an amount ranging from 0.01% to 10% by weight relative to the total weight of the dyeing composition.

31. The dyeing composition according to claim 28, further comprising at least one additional direct dye and/or at least one oxidation base optionally in combination with at least one coupler.

32. The composition according to claim 31, wherein the at least additional direct dye is a cationic or nonionic dye chosen from nitrobenzene dyes, azo, azomethine, methine, tetraazapentamethine, anthraquinone, naphthoquinone, benzoquinone, phenothiazine, indigoid, xanthene, phenanthridine, phthalocyanine dyes, dyes derived from triarylmethane, natural dyes, and mixtures thereof.

33. The composition according to claim 31, wherein the at least one oxidation base is chosen from p-phenylenediamines, bisphenylalkylenediamines, o-aminophenols, p-aminophenols and heterocyclic bases.

34. The composition according to claim 31, wherein the at least coupler is chosen from m-aminophenols, m-phenylenediamines, m-diphenols, naphthols, heterocyclic couplers, acid addition salts thereof, and mixtures thereof.

35. The composition according to claim 31, further comprising at least one oxidizing agent.

36. A method of coloring keratin fibers comprising contacting said fibers, dry or wet, with a dyeing composition for a time sufficient to give a desired color, wherein the dyeing composition comprises, in a medium appropriate for dyeing keratin fibers, at least one direct dye chosen from those of formulae (I), (II), (III) and (IV), acid addition salts thereof, and solvates thereof, wherein in the compounds of formulae (I), (II), (III) and (IV), the formula members attached to each side of the linker L are either compositionally different or positionally different such that the compounds as a whole are dissymmetrical: 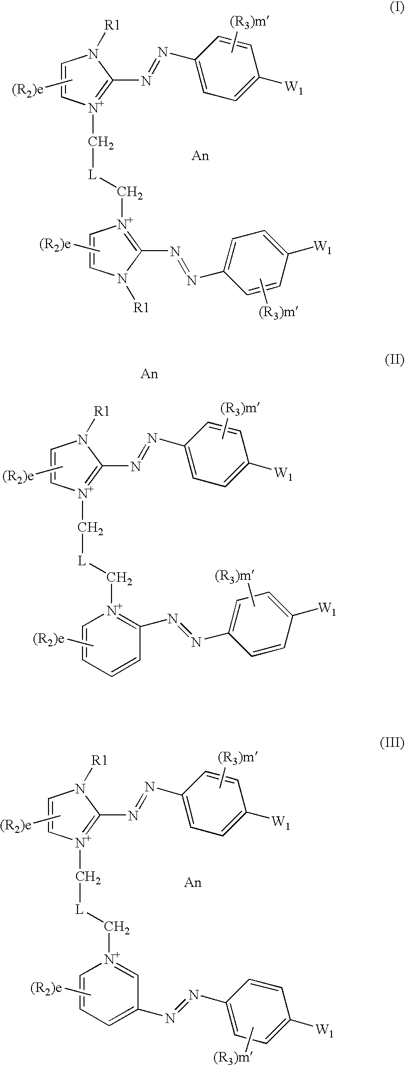 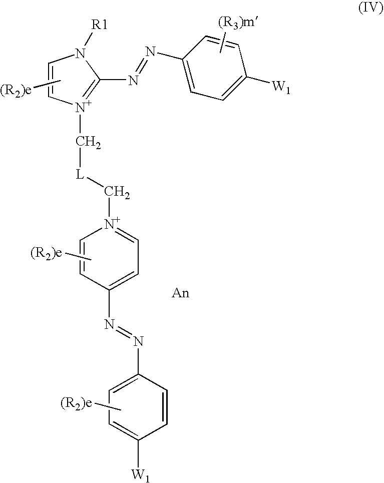
wherein:
the radicals R₁, which are identical or different, independently of one another are chosen from:
optionally substituted C₁-C₄ alkyl radicals;
optionally substituted phenyl radicals;
optionally substituted benzyl radicals;
the radicals R₂, which are identical or different, independently of one another are chosen from:
optionally substituted C₁-C₁₆ alkyl radicals optionally interrupted by at least one heteroatom and/or at least one group containing at least one heteroatom, said alkyl radical being further optionally substituted by at least one group chosen from thio (—SH), C₁-C₄ thioalkyl; C₁-C₄ alkylsulphinyl and C₁-C₄ alkylsulphonyl groups;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group, wherein the two alkyl radicals may optionally form, with the nitrogen atom to which they are attached, a heterocycle containing 1 to 3 heteroatoms chosen from N, O and S, and containing 5 to 7 ring members, wherein said heterocycle is saturated or unsaturated, aromatic or non-aromatic and is optionally substituted;
alkylcarbonylamino groups (RCO—NR′—) wherein R is a C₁-C₄ alkyl radical and R′ is chosen from hydrogen and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein R is a C₁-C₄ alkyl radical and R′ is chosen from a hydrogen atom and a C₁-C₄ alkyl radical;
optionally substituted aryl radicals;
optionally substituted (C₁-C₄)alkylaryl radicals;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
thio groups (HS—);
alkylthio groups (RS—) wherein R is an optionally substituted C₁-C₄ alkyl radical;
when e is 2, for each of the formulae (I), (II), (III) and (IV), two radicals R₂ may optionally form, with the carbon atoms to which they are attached, a secondary ring, aromatic or non-aromatic, containing 5 or 6 ring members, which is optionally substituted by at least one identical or different group chosen from hydroxyl, C₁-C₄ alkyl, C₁-C₄ alkoxy, C₂-C₄ (poly)hydroxyalkoxy, amino, and amino substituted by one or two identical or different C₁-C₄ alkyl radicals which optionally carry at least one hydroxyl group;
e is an integer ranging from 0 to 4; when e is less than 4, the unsubstituted carbon atom(s) of the heterocycle of the formulae (I), (II), (III) and (IV) carry a hydrogen atom,
the radicals R₃, from formulae (I), (II), (III) and (IV), which are identical or different, independently of one another are chosen from:
optionally substituted C₁-C₁₆ alkyl radicals optionally interrupted by at least one heteroatom or by at least one group containing at least one heteroatom;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group; wherein the two alkyl radicals may optionally form, with the nitrogen atom to which they are attached, a heterocycle containing 1 to 3 heteroatoms chosen from N, O and S, containing 5 to 7 ring members, and which is saturated or unsaturated, aromatic or non-aromatic and is optionally substituted;
alkylcarbonylamino groups (RCO—NR′—) wherein the radical R is a C₁-C₄ alkyl radical and the radical R′ is chosen from a hydrogen atom and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein radicals R and R′ independently of one another are chosen from a hydrogen atom and a C₁-C₄ alkyl radical;
thio groups (HS—);
alkylthio groups (RS—) wherein R is a C₁-C₄ alkyl radical;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
when m′ is greater than or equal to 2, two adjacent radicals R₃ may form, with the carbon atoms to which they are attached, a secondary ring, aromatic or non-aromatic containing 6 ring members, which is optionally substituted by at least one identical or different group chosen from: hydroxyl, C₁-C₄ alkyl, C₁-C₄ alkoxy, C₂-C₄ (poly)hydroxyalkoxy, C₁-C₄ alkylcarbonylamino, amino, and amino substituted by one or two identical or different C₁-C₄ alkyl radicals which optionally carry at least one hydroxyl group;
m′ is an integer from 0 to 4; when m′ is less than 4, then the unsubstituted carbon atom(s) of the aromatic ring of the formulae (I), (II), (III) and (IV) carry a hydrogen atom;
the W₁ groups of the formulae (I), (II), (III) and (IV), which are identical or different, independently of one another, are chosen from:
hydrogen atoms;
halogen atoms chosen from bromine, chlorine and fluorine;
a group chosen from —NR₅R₆, OR₇, —NR₄-Ph-NR₅R₆, —NR₄-Ph-OR₇, —O-Ph-OR₇ and —O-Ph-NR₅R₆ groups, wherein:
R₄ and R₇, which are identical or different, are chosen from hydrogen atoms, optionally substituted C₁-C₂₀ alkyl radicals, optionally substituted C₁-C₃ aralkyl radicals and optionally substituted phenyl radicals;
R₅ and R₆, which are identical or different, are chosen from hydrogen atoms, optionally substituted C₁-C₂₀ alkyl radicals, optionally substituted phenyl radicals, optionally substituted C₁-C₃ aralkyl radicals and alkylcarbonyl radicals (R—CO—) wherein R is a C₁-C₄ alkyl radical;
R₅ and R₆ may optionally form, with the nitrogen atom to which they are attached, a heterocycle containing 1 to 3 heteroatoms, chosen from N, O and S, and containing 5 to 7 ring members, which is saturated or unsaturated, aromatic or non-aromatic and is optionally substituted;
R₅ and R₆ may optionally form, with the carbon atom of the aromatic ring adjacent to that to which —NR₅R₆ is attached, a 5- or 6-membered saturated heterocycle;
Ph is an optionally substituted phenyl radical;
L is chosen from a cationic linker and a non-cationic linker;
An is chosen from at least one identical or different cosmetically acceptable anions which ensure the electroneutrality of the compounds of formula (I), (II), (III) and (IV).

37. A multi-compartment device, wherein a first compartment comprises a dyeing composition and a second compartment comprises an oxidizing composition, said dyeing composition comprising, in a medium appropriate for dyeing keratin fibers, at least one direct dye chosen from those of formulae (I), (II), (III) and (IV), acid addition salts thereof, and solvates thereof, wherein in the compounds of formulae (I), (II), (III) and (IV), the formula members attached to each side of the linker L are either compositionally different or positionally different such that the compounds as a whole are dissymmetrical: 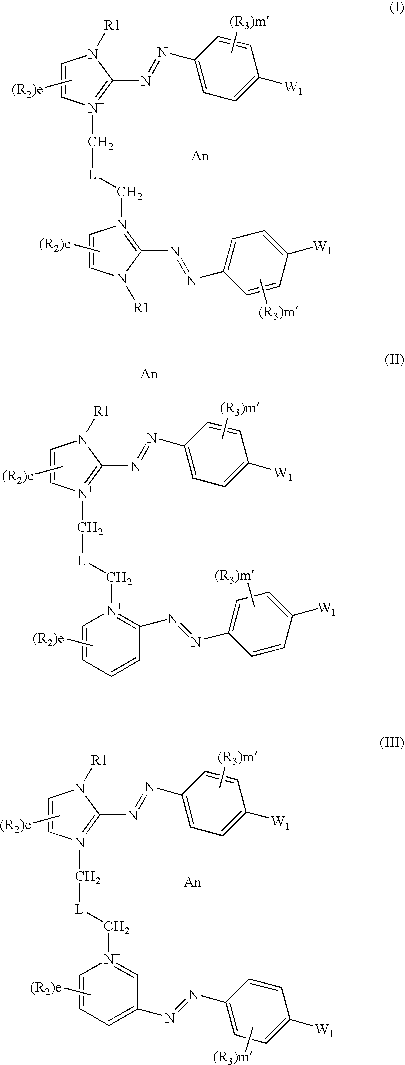 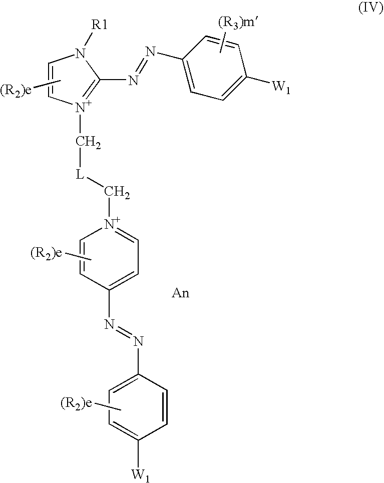
wherein:
the radicals R₁, which are identical or different, independently of one another are chosen from:
optionally substituted C₁-C₄ alkyl radicals;
optionally substituted phenyl radicals;
optionally substituted benzyl radicals;
the radicals R₂, which are identical or different, independently of one another are chosen from:
optionally substituted C₁-C₁₆ alkyl radicals optionally interrupted by at least one heteroatom and/or at least one group containing at least one heteroatom, said alkyl radical being further optionally substituted by at least one group chosen from thio (—SH), C₁-C₄ thioalkyl; C₁-C₄ alkylsulphinyl and C₁-C₄ alkylsulphonyl groups;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group, wherein the two alkyl radicals may optionally form, with the nitrogen atom to which they are attached, a heterocycle containing 1 to 3 heteroatoms chosen from N, O and S, and containing 5 to 7 ring members, wherein said heterocycle is saturated or unsaturated, aromatic or non-aromatic and is optionally substituted;
alkylcarbonylamino groups (RCO—NR′—) wherein R is a C₁-C₄ alkyl radical and R′ is chosen from hydrogen and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein R is a C₁-C₄ alkyl radical and R′ is chosen from a hydrogen atom and a C₁-C₄ alkyl radical;
optionally substituted aryl radicals;
optionally substituted (C₁-C₄)alkylaryl radicals;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
thio groups (HS—);
alkylthio groups (RS—) wherein R is an optionally substituted C₁-C₄ alkyl radical;
when e is 2, for each of the formulae (I), (II), (III) and (IV), two radicals R₂ may optionally form, with the carbon atoms to which they are attached, a secondary ring, aromatic or non-aromatic, containing 5 or 6 ring members, which is optionally substituted by at least one identical or different group chosen from hydroxyl, C₁-C₄ alkyl, C₁-C₄ alkoxy, C₂-C₄ (poly)hydroxyalkoxy, amino, and amino substituted by one or two identical or different C₁-C₄ alkyl radicals which optionally carry at least one hydroxyl group;
e is an integer ranging from 0 to 4; when e is less than 4, the unsubstituted carbon atom(s) of the heterocycle of the formulae (I), (II), (III) and (IV) carry a hydrogen atom,
the radicals R₃, from formulae (I), (II), (III) and (IV), which are identical or different, independently of one another are chosen from:
optionally substituted C₁-C₁₆ alkyl radicals optionally interrupted by at least one heteroatom or by at least one group containing at least one heteroatom;
hydroxyl groups;
C₁-C₄ alkoxy groups;
C₂-C₄ (poly)hydroxyalkoxy groups;
alkoxycarbonyl groups (RO—CO—) wherein R is a C₁-C₄ alkyl radical;
alkylcarbonyloxy groups (RCO—O—) wherein R is a C₁-C₄ alkyl radical;
alkylcarbonyl groups (R—CO—) wherein R is a C₁-C₄ alkyl radical;
amino groups;
amino groups substituted by one or two identical or different C₁-C₄ alkyl radicals optionally carrying at least one hydroxyl group; wherein the two alkyl radicals may optionally form, with the nitrogen atom to which they are attached, a heterocycle containing 1 to 3 heteroatoms chosen from N, O and S, containing 5 to 7 ring members, and which is saturated or unsaturated, aromatic or non-aromatic and is optionally substituted;
alkylcarbonylamino groups (RCO—NR′—) wherein the radical R is a C₁-C₄ alkyl radical and the radical R′ is chosen from a hydrogen atom and a C₁-C₄ alkyl radical;
aminocarbonyl groups ((R)₂N—CO—) wherein radicals R independently of one another are chosen from a hydrogen atom and a C₁-C₄ alkyl radical;
ureido groups (N(R)₂—CO—NR′—) wherein radicals R and R′ independently of one another are chosen from a hydrogen atom and a C₁-C₄ alkyl radical;
aminosulphonyl groups ((R)₂N—SO₂—) wherein radicals R independently of one another are chosen from a hydrogen atom and a C₁-C₄ alkyl radical;
alkylsulphonylamino groups (RSO₂—NR′—) wherein radicals R and R′ independently of one another are chosen from a hydrogen atom and a C₁-C₄ alkyl radical;
thio groups (HS—);
alkylthio groups (RS—) wherein R is a C₁-C₄ alkyl radical;
alkylsulphinyl groups (R—SO—) wherein R is a C₁-C₄ alkyl radical;
alkylsulphonyl groups (R—SO₂—) wherein R is a C₁-C₄ alkyl radical;
nitro groups;
cyano groups;
halogen atoms;
when m′ is greater than or equal to 2, two adjacent radicals R₃ may form, with the carbon atoms to which they are attached, a secondary ring, aromatic or non-aromatic containing 6 ring members, which is optionally substituted by at least one identical or different group chosen from: hydroxyl, C₁-C₄ alkyl, C₁-C₄ alkoxy, C₂-C₄ (poly)hydroxyalkoxy, C₁-C₄ alkylcarbonylamino, amino, and amino substituted by one or two identical or different C₁-C₄ alkyl radicals which optionally carry at least one hydroxyl group;
m′ is an integer from 0 to 4; when m′ is less than 4, then the unsubstituted carbon atom(s) of the aromatic ring of the formulae (I), (II), (III) and (IV) carry a hydrogen atom;
the W₁ groups of the formulae (I), (II), (III) and (IV), which are identical or different, independently of one another, are chosen from:
hydrogen atoms;
halogen atoms chosen from bromine, chlorine and fluorine;
a group chosen from —NR₅R₆, OR₇, —NR₄-Ph-NR₅R₆, —NR₄-Ph-OR₇, —O-Ph-OR₇ and —O-Ph-NR₅R₆ groups, wherein:
R₄ and R₇, which are identical or different, are chosen from hydrogen atoms, optionally substituted C₁-C₂₀ alkyl radicals, optionally substituted C₁-C₃ aralkyl radicals and optionally substituted phenyl radicals;
R₅ and R₆, which are identical or different, are chosen from hydrogen atoms, optionally substituted C₁-C₂₀ alkyl radicals, optionally substituted phenyl radicals, optionally substituted C₁-C₃ aralkyl radicals and alkylcarbonyl radicals (R—CO—) wherein R is a C₁-C₄ alkyl radical;
R₅ and R₆ may optionally form, with the nitrogen atom to which they are attached, a heterocycle containing 1 to 3 heteroatoms, chosen from N, O and S, and containing 5 to 7 ring members, which is saturated or unsaturated, aromatic or non-aromatic and is optionally substituted;
R₅ and R₆ may optionally form, with the carbon atom of the aromatic ring adjacent to that to which —NR R₆ is attached, a 5- or 6-membered saturated heterocycle;
Ph is an optionally substituted phenyl radical;
L is chosen from a cationic linker and a non-cationic linker;
An is chosen from at least one identical or different cosmetically acceptable anions which ensure the electroneutrality of the compounds of formula (I), (II), (III) and (IV).
